(19) United States
(12) Patent Application Publication
(10) Pub. No.: US 2025/0099423 A1
(43) Pub. Date: Mar. 27, 2025

(54) PIPERAZINE-BASED AGONISTS OF LFA-1 AND VLA-4

(71) Applicant: Texas Heart Institute, Houston, TX (US)

(72) Inventors: Robert V. MARKET, Houston, TX (US); Ronald J. BIEDIGER, Houston, TX (US); Darren G. WOODSIDE, Houston, TX (US)

(73) Assignee: Texas Heart Institute, Houston, TX (US)

(21) Appl. No.: 18/682,681

(22) PCT Filed: Jun. 9, 2022
(86) PCT No.: PCT/US2022/032896

§ 371 Date: Feb. 9, 2024

Related U.S. Application Data
(60) Provisional application No. 63/231,549, filed on Aug. 10, 2021.

(51) Int. Cl. A61K 31/381; A61K 31/166; A61K 31/27; C07C 237/24; C07C 271/24; C07D 409/12; C07D 409/14

(52) U.S. Cl. CPC A61K 31/381; A61K 31/166; A61K 31/27; C07C 237/24; C07C 271/24; C07D 409/12; C07D 409/14

(57) ABSTRACT

N,N-disubstituted aminocarbonyl compounds and their use as integrin agonists for enhancing the binding of integrin-expressing cells to integrin-binding ligands or receptors.

### CROSS-REFERENCE TO RELATED APPLICATIONS

This application is a National Phase Entry into the U.S. under 35 U.S.C. § 371 of and claims priority to PCT Application No. PCT/US2022/032896, filed Jun. 9, 2022, entitled “PIPERAZINE-BASED AGONISTS OF LFA-1 AND VLA-4,” which claims priority to U.S. Provisional Application Ser. No. 63/231,549 filed Aug. 10, 2021 and entitled “PIPERAZINE-BASED AGONISTS OF LFA-1 AND VLA-4” by Market et al., which is incorporated herein by reference in its entirety.

### STATEMENT REGARDING FEDERALLY SPONSORED RESEARCH OR DEVELOPMENT

This invention was made with Government support under grant numbers 2 R42 CA203456-02A1, and 1R41 AI145507-01 awarded by the National Cancer Institute, and the National Institutes of Health, respectively. The government has certain rights in the invention.

### BACKGROUND

### Field of the Disclosure

The present disclosure generally relates to N,N-disubstituted aminocarbonyl compounds and to their use as integrin agonists for enhancing binding of integrin-expressing cells to integrin-binding ligands or receptors. These may be useful in cell-based therapies as well as adjuvants for vaccines and cancer therapies among other uses. Potency against VLA-4 is generally in the same range as other more lipophilic classes of previously reported integrin agonist compounds. Compounds also exhibit enhanced activity against lymphocyte function-associated antigen-1 or LFA-1 agonism.

### Background of the Disclosure

Integrin agonists of the prior art are known to activate integrins by augmenting interior cell mechanisms or upregulating integrin gene expression or permanently damaging or chemically altering the integrins. There is however a need in the art for integrin activating compounds that do not permanently damage or chemically alter their target integrins. This lack of permanent damage or chemical alteration of the integrins is critical for stem cell, immune cell, and other cells used in cell based therapies. Hence there is a need in the art for integrin activating compounds that facilitate activating, priming, homing, trafficking, infiltrating, targeting, and/or other cell movements of cells through the body of a patient, wherein the natural functioning of the cell is not permanently damaged or chemically altered, only enhanced.

### BRIEF SUMMARY OF DISCLOSURE

Disclosed herein, in one embodiment is a compound of Formula I,

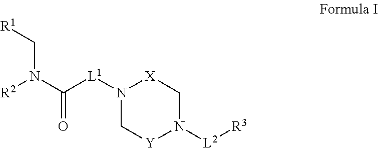

wherein R¹ is an aryl ring; R2 is selected from aryl, aralkyl, and lower alkyl; L¹ is selected from —(CH₂)ₙ—, —O(CH₂)ₙ—; L² is selected from, —CO—, —CO(CH₂)ₘ, —COO(CH₂)ₘ—, —(CH₂)ₘ—, —(CH₂)ₘO—; R³ is selected from aryl, heterocyclyl, CONR⁴R⁵, and —COR⁶; X and Y are independently selected from —CH₂— and —C(O)—; n is an integer of from 1 to 4; m, when present, is an integer of from 1 to 2; R⁴ and R⁵, when present, are independently selected from the group of lower alkyl and aralkyl; R⁶, when present, is a heterocyclic ring; when present, each R¹ and R² may be unsubstituted or substituted with a substituent selected from the group consisting essentially of lower alkyl, alkoxy, hydroxyalkyl, —OH, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, and heterocyclylalkyl, groups; R³, R⁴, R⁵ and R⁶ may be unsubstituted or substituted with a substituent selected from the group consisting essentially of hydroxy, alkoxy, dialkylamino, halogen, lower alkyl, hydroxyalkyl, aliphatic acyl, —CF₃, oxo, —CN, alkoxyalkyl, (C₁-C₃ alkyl)₂amino, alkoxyalkoxy, cycloalkyl, cycloalkylalkyl, aryl, heterocyclyl, alkylaryl, aralkyl, alkylheterocyclyl, heterocyclylalkyl, and aryloxyalkyl groups; and pharmaceutically acceptable salts thereof.

In another embodiment, a composition comprising a compound of Formula I wherein R¹ is selected from the group consisting essentially of substituted phenyl, and substituted or unsubstituted thienyl, oxazolyl, isoxazolyl, pyrrolyl andpyridylis disclosed.

In a further embodiment, R³ is selected from the group consisting essentially of:

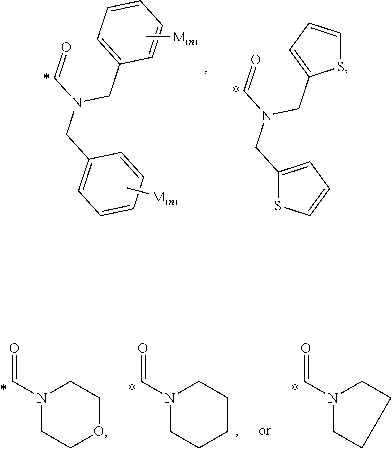

wherein the asterisk * represents the attachment to L²; each M is selected from the groups consisting essentially of hydroxy, alkoxy, dialkylamino, halogen, and alkyl; and each n is an integer from 1-2.

In a still further embodiment, the compound of Formula I, is selected from the group consisting essentially of 2,2′-((piperazine-1,4-diylbis(ethane-2,1-diyl))bis(oxy))bis(N,N-bis(thiophen-2-ylmethyl)acetamide), piperazine-1,4-diylbis(ethane-2,1-diyl) bis(bis(3-methoxybenzyl)carbamate); benzyl 4-(2-((4-(dimethylamino)benzyl)(4-methoxybenzyl)amino)-2-oxoethyl)piperazine-1-carboxylate; 2,2′-(2,5-dioxopiperazine-1,4-diyl) bis(N,N-bis(thiophen-2-ylmethyl)acetamide); benzyl 4-(2-((4-hydroxybenzyl)(4-methoxybenzyl)amino)-2-oxoethyl)piperazine-1-carboxylate; benzyl 4-(2-((4-(dimethylamino)benzyl)(4-hydroxybenzyl)amino)-2-oxoethyl)piperazine-1-carboxylate dihydrochloride; benzyl 4-(2-(bis(4-hydroxybenzyl)amino)-2-oxoethyl)piperazine-1-carboxylate; benzyl 4-(2-(bis(4-methoxybenzyl)amino)-2-oxoethyl)piperazine-1-carboxylate; benzyl 4-(2-(bis(3-methoxybenzyl)amino)-2-oxoethyl)piperazine-1-carboxylate; piperazine-1,4-diylbis(ethane-2,1-diyl) bis(4-(dimethylamino)benzyl (4-methoxybenzyl)carbamate); 2,2′-(2,5-dioxopiperazine-1,4-diyl)bis(N-(3-(dimethylamino)benzyl)-N-(4-methoxybenzyl)acetamide); 2,2′-(2,5-dioxopiperazine-1,4-diyl) bis(N,N-bis(3-methoxybenzyl)acetamide); 2,2′-(piperazine-1,4-diyl)bis(N,N-bis(4-methoxybenzyl)acetamide); benzyl 4-(4-(bis(4-methoxybenzyl)amino)-4-oxobutyl)piperazine-1-carboxylate; benzyl 4-(5-(bis(thiophen-2-ylmethyl)amino)-5-oxopentyl)piperazine-1-carboxylate; 2,2′-(piperazine-1,4-diyl)bis(N,N-bis(thiophen-2-ylmethyl)acetamide); 3-methoxybenzyl 4-(2-(bis(4-methoxybenzyl)amino)-2-oxoethyl)piperazine-1-carboxylate; benzyl 4-(5-(bis(3-methoxybenzyl)amino)-5-oxopentyl)piperazine-1-carboxylate; benzyl 4-(5-(bis(4-methoxybenzyl)amino)-5-oxopentyl)piperazine-1-carboxylate; 4-methoxyphenethyl 4-(2-(bis(4-methoxybenzyl)amino)-2-oxoethyl)piperazine-1-carboxylate; 2,2′-(2-oxopiperazine-1,4-diyl)bis(N,N-bis(4-methoxybenzyl)acetamide); 3-methoxybenzyl 4-(2-((4-(dimethylamino)benzyl)(4-methoxybenzyl)amino)-2-oxoethyl)-3-oxopiperazine-1-carboxylate; N,N-bis(4-methoxybenzyl)-2-(4-(3-methoxybenzyl)-2-oxopiperazin-1-yl)acetamide; N-(4-(dimethylamino)benzyl)-2-(4-(3-methoxybenzoyl)-2-oxopiperazin-1-yl)-N-(4-methoxybenzyl)acetamide; 2-(4-(3-methoxybenzoyl)-2-oxopiperazin-1-yl)-N,N-bis(4-methoxybenzyl)acetamide; N,N-bis(4-methoxybenzyl)-2-(4-(2-(3-methoxyphenyl)acetyl)-2-oxopiperazin-1-yl)acetamide; (2,5-dioxopiperazine-1,4-diyl)bis(ethane-2,1-diyl) bis(bis(3-methoxybenzyl)carbamate); (2,5-dioxopiperazine-1,4-diyl)bis(ethane-2,1-diyl) bis(bis(4-methoxybenzyl)carbamate); (2,5-dioxopiperazine-1,4-diyl)bis(ethane-2,1-diyl) bis(4-(dimethylamino)benzyl (3-methoxybenzyl)carbamate); (2,5-dioxopiperazine-1,4-diyl)bis(ethane-2,1-diyl)bis(4-(dimethylamino)benzyl (4-methoxybenzyl)carbamate); (2,5-dioxopiperazine-1,4-diyl)bis(ethane-2,1-diyl) bis(bis(thiophen-2-ylmethyl)carbamate); 2,2′-(piperazine-1,4-diyl)bis(N-isobutyl-N-(4-methoxybenzyl)acetamide); 2,2′-(2-oxopiperazine-1,4-diyl)bis(N-isobutyl-N-(4-methoxybenzyl)acetamide); 2,2′-(2,5-dioxopiperazine-1,4-diyl)bis(N-isobutyl-N-(4-methoxybenzyl)acetamide); 2,2′-(2-oxopiperazine-1,4-diyl)bis(N-(4-(dimethylamino)benzyl)-N-(4-methoxybenzyl)acetamide); and 2,2′-(2,5-dioxopiperazine-1,4-diyl)bis(N,N-bis(4-(dimethylamino)benzyl)acetamide). In some embodiments disclosed herein is a pharmaceutical composition comprising a compound of Formula I or a pharmaceutically acceptable salt thereof; and a pharmaceutically acceptable carrier.

### DETAILED DESCRIPTION OF DISCLOSED EXEMPLARY EMBODIMENTS

The following discussion is directed to various exemplary embodiments. However, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and that the scope of this disclosure, including the claims, is not limited to that embodiment.

The drawing figures are not necessarily to scale. Certain features and components herein may be shown exaggerated in scale or in somewhat schematic form and some details of conventional elements may be omitted in interest of clarity and conciseness.

In the following discussion and in the claims, the terms “including” and “comprising” are used in an open-ended fashion, and thus should be interpreted to mean “including, but not limited to . . . ” As used herein, the term “about,” when used in conjunction with a percentage or other numerical amount, means plus or minus 10% of that percentage or other numerical amount. For example, the term “about 80%,” would encompass 80% plus or minus 8%. References cited herein are incorporated in their entirety by such reference.

### Definitions

In addition to having their customary and usual meaning, the following definitions apply where the context permits in the specification and claims:

“Pharmaceutical composition” refers to a mixture of one or more chemicals, or pharmaceutically acceptable salts thereof, with a suitable carrier, for administration to a mammal as a medicine.

“Therapeutically effective amount” refers to that amount of the compound being administered that will relieve at least to some extent one or more of the symptoms of the disorder being treated. For example, an amount of the compound effective to prevent, alleviate or ameliorate symptoms of disease or prolong the survival of the subject being treated.

With respect to a disease or disorder, the term “treatment” refers to preventing, deterring the occurrence of the disease or disorder, arresting, regressing, or providing relief from symptoms or side effects of the disease or disorder and/or prolonging the survival of the subject being treated.

The term “alkyl” as used herein alone or in combination refers to C₁-C₁₂ straight or branched, substituted or unsubstituted saturated chain radicals derived from saturated hydrocarbons by the removal of one hydrogen atom. Representative examples of alkyl groups include methyl, ethyl, n-propyl, iso-propyl, n-butyl, sec-butyl, iso-butyl, and tert-butyl among others.

The term “alkenyl”, alone or in combination, refers to a substituted or unsubstituted straight-chain or substituted or unsubstituted branched-chain alkenyl radical containing from 2 to 10 carbon atoms. Examples of such radicals include, but are not limited to, ethenyl, E- and Z-pentenyl, decenyl and the like.

The term “lower” modifying “alkyl”, “alkenyl”, or “alkoxy” refers to a C₁-C₆ unit for a particular functionality. For example, lower alkyl means a C₁-C₆ alkyl.

The term “cycloalkyl” as used herein alone or in combination refers to a substituted or unsubstituted aliphatic ring system having 3 to 10 carbon atoms and 1 to 3 rings, including, but not limited to cyclopropyl, cyclopentyl, cyclohexyl, norbornyl, and adamantyl among others. Cycloalkyl groups can be unsubstituted or substituted with one, two or three substituents independently selected from lower alkyl, haloalkyl, alkoxy, thioalkoxy, amino, alkylamino, dialkylamino, hydroxy, halo, mercapto, nitro, carboxaldehyde, carboxy, alkoxycarbonyl and carboxamide. This term is meant to encompass cycloalkenyl groups. “Cycloalkyl” includes cis or trans forms. Furthermore, the substituents may either be in endo or exo positions in the bridged bicyclic systems.

The term “cycloalkenyl” as used herein alone or in combination refers to a cyclic carbocycle containing from 4 to 8 carbon atoms and one or more double bonds. Examples of such cycloalkenyl radicals include, but are not limited to, cyclopentenyl, cyclohexenyl, cyclopentadienyl and the like.

The term “cycloalkylalkyl” as used herein refers to a cycloalkyl group appended to a lower alkyl radical, including, but not limited to cyclohexyl methyl.

The term “halo” or “halogen” as used herein refers to I, Br, Cl or F.

The term “haloalkyl” as used herein refers to a lower alkyl radical, to which is appended at least one halogen substituent, for example chloromethyl, fluoroethyl, trifluoromethyl and pentafluoroethyl among others.

The term “alkoxy”, alone or in combination, refers to a radical of the formula alkyl-O—, wherein the term “alkyl” is as defined above. Examples of suitable alkyl ether radicals include, but are not limited to, methoxy, ethoxy, n-propoxy, iso-propoxy, n-butoxy, iso-butoxy, sec-butoxy, tert-butoxy and the like.

The term “alkenoxy”, alone or in combination, refers to a radical of formula alkenyl-O—, provided that the radical is not an enol ether, wherein the term “alkenyl” is as defined above. Examples of suitable alkenoxy radicals include, but are not limited to, allyloxy, E- and Z-3-methyl-2-propenoxy and the like.

The term “alkynoxy”, alone or in combination, refers to a radical of formula alkynyl-O—, provided that the radical is not an -ynol ether. Examples of suitable alkynoxy radicals include, but are not limited to, propargyloxy, 2-butynyloxy and the like.

The term “carboxyl” as used herein refers to —CO₂ H.

The term “thioalkoxy”, refers to a thioether radical of formula alkyl-S—, wherein “alkyl” is as defined above.

The term “carboxaldehyde” as used herein refers to —C(O)R wherein R is hydrogen.

The term “carboxamide” as used herein refers to —C(O)NR₂ wherein R is hydrogen, alkyl or any other suitable substituent.

The term “alkoxyalkoxy” as used herein refers to R_{b}O—R_{c}O— wherein R_{b} is lower alkyl as defined above and Rₑ is alkylene wherein alkylene is —(CH₂)_{n′}— wherein n′ is an integer from 1 to 6. Representative examples of alkoxyalkoxy groups include methoxymethoxy, ethoxymethoxy, and t-butoxymethoxy among others.

The term “alkylamino” as used herein refers to R_{d}NH— wherein R_{d} is a lower alkyl group, for example, ethylamino, butylamino, among others.

The term “alkenylamino” alone or in combination, refers to a radical of formula alkenyl-NH— or (alkenyl)₂ N—, wherein the term “alkenyl” is as defined above, provided that the radical is not an enamine. An example of such alkenylamino radicals is the allylamino radical.

The term “dialkylamino” as used herein refers to RₑR_{f}N— wherein Rₑ and R_{f} are independently selected from lower alkyl, for example diethylamino, and methyl propylamino, among others.

The term “amino” as used herein refers to H₂N—.

The term “alkoxycarbonyl” as used herein refers to an alkoxyl group as previously defined appended to the parent molecular moiety through a carbonyl group. Examples of alkoxycarbonyl include methoxycarbonyl, ethoxycarbonyl, and isopropoxycarbonyl among others.

The term “aryl” or “aromatic” as used herein alone or in combination refers to a substituted or unsubstituted carbocyclic aromatic group having about 6 to 12 carbon atoms such as phenyl, naphthyl, indenyl, indanyl, azulenyl, fluorenyl and anthracenyl; or a heterocyclic aromatic group selected from the group consisting of furyl, thienyl, pyridyl, pyrrolyl, oxazolyl, thiazolyl, imidazolyl, pyrazolyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthyridinyl, pteridinyl, carbazolyl, acridinyl, phenazinyl, phenothiazinyl, phenoxazinyl, pyrazolo[1,5-c]triazinyl and the like. “Arylalkyl” and “alkylaryl” employ the term “alkyl” as defined above. Rings may be multiply substituted. Aromatic rings may be fused with other aromatic or non-aromatic rings to form multicyclic rings, and are also encompassed by the term “aromatic,” as used herein.

The term “aralkyl”, alone or in combination, refers to an aryl substituted alkyl radical, wherein the terms “alkyl” and “aryl” are as defined above. Examples of suitable aralkyl radicals include, but are not limited to, phenylmethyl, phenethyl, phenylhexyl, diphenylmethyl, pyridylmethyl, tetrazolyl methyl, furylmethyl, imidazolyl methyl, indolylmethyl, thienylpropyl and the like.

The term “aralkenyl”, alone or in combination, refers to an aryl substituted alkenyl radical, wherein the terms “aryl” and “alkenyl” are as defined above.

The term “arylamino”, alone or in combination, refers to a radical of formula aryl-NR-g-, wherein “aryl” is as defined above. Rg may be selected from the group consisting of H, lower alkyl, aryl and aralkyl among others. Examples of arylamino radicals include, but are not limited to, phenylamino (anilido), naphthlamino, 2-, 3-, and 4-pyridylamino and the like.

The term “biaryl”, alone or in combination, refers to a radical of formula aryl-aryl, wherein the term “aryl” is as defined above.

The term “thioaryl”, alone or in combination, refers to a radical of formula aryl-S—, wherein the term “aryl” is as defined above. An example of a thioaryl radical is the thiophenyl radical.

The term “aroyl” or “aromatic acyl”, alone or in combination, refers to a radical of formula aryl-CO—, wherein the term “aryl” is as defined above. Examples of suitable aromatic acyl radicals include, but are not limited to, benzoyl, 4-halobenzoyl, 4-carboxybenzoyl, naphthoyl, pyridylcarbonyl and the like.

The term “aliphatic acyl”, alone or in combination, refers to a radical of formula alkyl-CO—, wherein the term “alkyl” is as defined above. Examples of suitable alkyl acyl radicals include, but are not limited to, acetyl, propionyl, isobutyryl, and the like.

The term “heterocyclyl”, alone or in combination, refers to a non-aromatic 3- to 10-membered ring containing at least one endocyclic N, O, or S atom. The heterocycle may be optionally aryl-fused. The heterocycle may also optionally be substituted with at least one substituent which is independently selected from the group consisting of halogen, hydroxyl, amino, nitro, trifluoromethyl, trifluoromethoxy, alkyl, aralkyl, alkenyl, aryl, cyano, carboxyl, alkoxycarbonyl, carboxyalkyl, oxo, arylsulfonyl and aralkylaminocarbonyl among others.

The term “alkylheterocyclyl” as used herein refers to an alkyl group as previously defined appended to the parent molecular moiety through a heterocyclyl group.

The term “heterocyclylalkyl” as used herein refers to a heterocyclyl group as previously defined appended to the parent molecular moiety through an alkyl group.

The term “aminal” as used herein refers to a hemi-acetal of the structure RCH(NH₂)(OH).

The terms “electron-withdrawing” or “electron-donating” refer to the ability of a substituent to withdraw or donate electrons relative to that of hydrogen if hydrogen occupied the same position in the molecule. These terms are well-understood by one skilled in the art and are discussed in ADVANCED ORGANIC CHEMISTRY by J. March 1985, pp. 16-18, incorporated herein by reference. Electron withdrawing groups include halo, nitro, carboxyl, lower alkenyl, lower alkynyl, carboxaldehyde, carboxyamido, aryl, quaternary ammonium, trifluoromethyl, and aryl lower alkanoyl among others. Electron donating groups include such groups as hydroxy, lower alkyl, amino, lower alkylamino, di(lower alkyl)amino, aryloxy, mercapto, lower alkylthio, lower alkylmercapto, and disulfide among others. One skilled in the art will appreciate that the aforesaid substituents may have electron donating or electron withdrawing properties under different chemical conditions. Moreover, the present invention contemplates any combination of substituents selected from the above-identified groups.

The most preferred electron donating or electron withdrawing substituents are halo, nitro, alkanoyl, carboxaldehyde, arylalkanoyl, aryloxy, carboxyl, carboxamide, cyano, sulfonyl, sulfoxide, heterocyclyl, guanidine, quaternary ammonium, lower alkenyl, sulfonium salts, hydroxy, lower alkoxy, lower alkyl, amino, lower alkylamino, di(lower alkyl)amino, amine lower alkyl mercapto, mercaptoalkyl, alkylthio and alkyldithio.

Use of the above terms is meant to encompass substituted and unsubstituted moieties. Substitution may be by one or more groups such as alcohols, ethers, esters, amides, sulfones, sulfides, hydroxyl, nitro, cyano, carboxy, amines, heteroatoms, lower alkyl, lower alkoxy, lower alkoxycarbonyl, alkoxyalkoxy, acyloxy, halogens, trifluoromethoxy, trifluoromethyl, alkyl, aralkyl, alkenyl, alkynyl, aryl, cyano, carboxy, carboalkoxy, carboxyalkyl, cycloalkyl, cycloalkylalkyl, heterocyclyl, alkylheterocyclyl, heterocyclylalkyl, oxo, arylsulfonyl and aralkylaminocarbonyl or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)O—. Rings may be substituted multiple times.

The term “mammals” includes humans and other animals.

The term “heteroatom” as used herein encompasses nitrogen, sulfur and oxygen.

The term “alpha” as used herein indicates the position immediately adjacent to the position described.

The term “one or a plurality”, “at least one” and “one or more” mean one item or multiple items (two or more).

The term “about” means that a value of a given quantity is within ±20% of the stated value. In other embodiments, the value is within ±15% of the stated value. In other embodiments, the value is within ±10% of the stated value. In other embodiments, the value is within ±5% of the stated value. In other embodiments, the value is within ±2.5% of the stated value. In other embodiments, the value is within ±1% of the stated value.

The term “substantially” means that a value of a given quantity is within ±10% of the stated value. In other embodiments, the value is within ±5% of the stated value. In other embodiments, the value is within ±2.5% of the stated value. In other embodiments, the value is within ±1% of the stated value. For some applications, a disclosed compound is administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. In some embodiments, a composition in liposome form contains, in addition to a disclosed agonist compound, stabilizers, preservatives, excipients and the like. The preferred lipids are natural and synthetic phospholipids and phosphatidyl cholines (lecithins) used separately or together. Methods of forming liposomes are known in the art. See, for example, Prescott, Ed., Methods in cell Biology, Volume XIV, Academic Press, New York, N.Y. (1976), p. 33 et seq.

The term “pharmaceutically acceptable pro-drugs” as used herein represents those pro-drugs of the disclosed compounds which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the disclosed compounds. Pro-drugs according to certain embodiments may be rapidly transformed in vivo to the parent compound of the above formula, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversable Carriers in Drug Design, American Pharmaceutical Association and Pergamon Press (1987), hereby incorporated by reference.

### Abbreviations

The following abbreviations are used herein:
Ac acetyl
AcOH acetic acid
Bn benzyl
Boc tert-butyloxycarbonyl
Cbz benzyloxycarbonyl
CDI N,N′-carbonyldiimidazole
DCE 1,2-dichloroethane
DCM dichloromethane (methlyene chloride)
dioxane 1,4-dioxane
DIPEA N,N-diisopropylethylamine
DMF N,N-dimethylformamide
DMSO dimethylsulfoxide
Et ethyl
EtOH ethanol
HBTU O-(Benzotriazol-1-yl)-N,N,N′,N′-tetramethyluronium hexafluorophosphate
Log P Log of the octanol water partition co-efficient
Me methyl
MeOH methanol
MS Mass Spectroscopy
m/z mass to charge ratio
NMR Nucluar Magnetic Resonance
OAc acetate
Ph phenyl
RT room temperature
tBu tert-butyl
TEA triethylamine
THF tetrahydrofuran
Tol toluene
Z benzyloxycarbonyl

In some embodiments, disclosed herein, small molecule compounds which enhance integrin-mediated adhesion may be beneficial as therapeutic agents, and in further embodiments said compounds may prove useful in the treatment of diseases or conditions that are amenable to cell-based therapy. Non-limiting examples of such diseases or conditions are myocardial infarction, heart failure, peripheral arterial disease, diabetes, renal failure, systemic lupus erythematosus, multiple sclerosis, pulmonary fibrosis, pulmonary hypertension, acute respiratory distress syndrome, Alzheimer's disease, Huntington's disease, Parkinson's disease, spinal cord injury, infertility, and bone marrow transplant. Accordingly, a group of chemical compounds which enhance the integrin-mediated binding of cells to their respective ligands are disclosed. Integrins targeted by these compounds include, but are not limited to, α4β1, α4β7, α5β1, and αLβ2. Corresponding ligands include, but are not limited to, VCAM-1, fibronectin, MAdCAM-1, ICAM-1, and ICAM-2.

Agonist compounds, the ability of representative compounds to enhance binding of integrin-expressing cells, and therapeutic applications of agonist-treated cells are further described as follows.

While not being bound to any theory of action, the present integrin agonists are believed to bind to certain integrins and to affect a change in the integrins from their inactive state to their active state. The change is thought to involve a change in conformation of the integrins from a closed configuration to an open configuration. The present integrin agonists, while activating the integrins, are displaced from the integrins when the integrins bind to their natural ligands involved in cell to cell adhesion. As opposed to integrin activating compounds that activate integrins by augmenting interior cell mechanisms or up regulate integrin gene expression or permanently damage or chemically alter the integrins, the present integrin activating compounds do not permanently damage or chemically alter their target integrins. This lack of permanently damage or chemically alteration of the integrins is critical for stem cell, immune cell, other cells used in cell based therapies as the present integrin activating compounds only facilitate activating, priming, homing, trafficking, infiltrating, targeting, and/or other cell movements of cells through the body of a patient, wherein the natural functioning of the cell is not permanently damage or chemically alter only enhanced.

### Agonist Pre-Treated Cells

One or more integrin-expressing cells are first treated (pre-treated) with an agonist compound having the general Formula I, as described herein, to form agonist-bound integrin molecules on the cell's surface. The integrin-expressing cells may be embryonic stem cells, adult stem/progenitor cells, or induced pluripotent stem cells, for example. In some embodiments, the cells express one or more of the integrins α4β1, α5β1, α4β7, and αLβ2. The treatment of the cells generally includes contacting the integrin-expressing cells in vitro with the agonist. In most applications the agonist compound in present in the treatment media at a concentration in the range of about 100 nM to about 30 μM. In some embodiments the agonist concentration is in the range of about 1 μM to about 10 μM. After exposure to the agonist, the resulting agonist-treated cells have an enhanced ability to bind to a cognate ligand. The integrin is expressed on the surface of the cells, and may be either naturally occurring or transgenically expressed by a cell that has been transformed to express an exogenous integrin gene. The protein or other cognate ligand to which the integrin binds is expressed either on a cell surface or is part of the extracellular matrix.

### Enhanced Binding of Pre-Treated Cells to Integrin-Binding Ligands

The agonist, as described herein, dissolved in a pharmaceutically acceptable diluent, is added to cell culture media or cell suspension and mixed. The resulting agonist-treated cells are introduced to an integrin-binding ligand or binding site, whereupon the treated cells bind, attach or adhere to the cognate ligands in solution, or on a surface or target tissue. In some embodiments an integrin binding protein is a vascular cell adhesion molecule-1 (VCAM 1), fibronectin, mucosal addressing cell adhesion molecule-1 (MAdCAM-1), intercellular adhesion molecule-1 (ICAM-1), or intercellular adhesion molecule-2 (ICAM-2). As a result of the agonist treatment, the binding of the agonist-treated cells to the ligand is enhanced or increased compared to binding of integrin-expressing cells not treated with the agonist. In some embodiments, at least 3 fold more agonist-treated cells are bound to a ligand-coated surface than untreated integrin-expressing cells. In some embodiments, up to 3 fold more agonist-treated cells than untreated cells are bound to an integrin binding protein.

### Enhanced Retention of Pre-Treated Cells to Tissues Expressing Integrin-Binding Ligands

Regardless of the cell type, mechanism of action, or how they are delivered, for many applications it is critical that the cells home to, and are retained in, a relevant injured tissue. Low levels of cell retention observed in animal models and clinical trials are considered one of the major impediments to the progress of cell-based therapies. Even when cells are injected locally, less than 10% of injected cells are typically retained after one hour and this number decreases over time in conventional cell-based therapies. The retention rates are even lower when delivered systemically. By comparison, many embodiments of the presently disclosed methods increase the rate of retention of exogenously delivered cells and will potentially greatly further efforts in regenerative medicine.

A method of enhancing retention of exogenously-introduced cells at an in vivo target site in a mammal generally includes (a) treating integrin-expressing cells in vitro with an agonist of integrin, wherein the agonist is a compound having the general Formula I, as described herein; (b) introducing the agonist-treated cells to an in vivo target site in the mammal; and (c) causing a greater number of said introduced agonist-treated cells to remain at said target site relative to the number of cells retained if integrin-expressing cells not treated with said agonist were introduced to said target site. The target site includes an integrin binding protein such as vascular cell adhesion molecule-1 (VCAM 1), fibronectin, mucosal addressin cellular adhesion molecule-1 (MAdCAM-1), inter-cellular adhesion molecule-1 (ICAM-1), or inter-cellular adhesion molecule-2 (ICAM-2), for example.

### Therapeutic Treatment of Damaged or Diseased Vascular Tissue

Agonist-treated cells prepared as described above are administered to a damaged or diseased vascular site in a vessel of a mammal. The cells are injected directly into, or around a site of damaged or diseased vascular tissue, as often occurs in tissue due to ischemia following a heart attack or in peripheral arterial disease. Alternatively, in some embodiments the agonist-treated cells are injected intravenously for homing to a damaged or diseased site where treatment is desired. The damaged or diseased tissue contains cells (e.g. endothelial cells) that express VCAM-1, and in which VCAM-1 exists on the cell surface. Expression of VCAM-1 is induced in some embodiments by inflammatory cytokines such as tumor necrosis factor-α, interleukin-4 and interleukin-1B. In some instances, cells or extracellular matrix at or adjacent to a treatment site express and bear on their surface one or more other integrin-binding protein such as fibronectin, mucosal addressing cellular adhesion molecule-1 (MAdCAM-1), intercellular adhesion molecule-1 (ICAM-1), or intercellular adhesion molecule-2 (ICAM-2). In those instances, the injected agonist-treated cells adhere to the cognate ligands at the damaged or diseased tissue site, causing a greater number of the administered agonist-treated cells to remain at the treatment site compared to the number of untreated integrin-expressing cells that would be retained if administered instead. The agonist-treated cells retained at the treatment site are allowed to grow and/or release paracrine factors, to regenerate vascular tissue at the damaged or diseased site, e.g., damage due to ischemia, autoimmune reactions, or mechanical injury. Paracrine factors are substances released from a cell that have effects on a neighboring cell, such as growth factors or cytokines.

### Cell-Based Therapies for Treatment of Other Diseases and Conditions

Use of the above-described agonist-treated cells for treatment of a number of diseases or conditions that are amenable to cell-based therapy is also contemplated in various embodiments. For example, myocardial infarction, peripheral artery disease, diabetes, renal failure, systemic lupus erythematosus, multiple sclerosis, pulmonary fibrosis, pulmonary hypertension, acute respiratory distress syndrome, Alzheimer's disease, Huntington's disease, Parkinson's disease, spinal cord injury, infertility, bone marrow transplant, cord blood transplant, and cancer immunotherapies, including CAR-T cell therapy are treated in some embodiments by injecting an above-described cell suspension intravenously, intraarterially, or directly in or around the injured area. New tissue is generated either by proliferation and differentiation of the injected cells and/or release of paracrine factors by the injected cells which induce proliferation and differentiation of neighboring host cells.

### Integrin Agonist Treating Compositions

Embodiments of this disclosure include compositions including one or more integrin agonist compounds of Formula I for treating cancer, especially treating solid tumor, wherein the one or more integrin agonist compounds of Formula I enhance homing, infiltration, engrafting, and invasion of natural T cell and/or treated and/or untreated effector cells and are present in a patient's blood and/or at a site of treatment in effective concentration of between about 1 fM and about 300 μM.

### Integrin Agonist and Stem/Progenitor Cell Treating Compositions

Embodiments of this disclosure include compositions including one or more integrin agonist compounds of Formula I and one or more stem/progenitor cells for stem cell therapies, especially treating solid tumor, wherein the one or more integrin agonist compounds of Formula I enhance homing, infiltration, engrafting, and invasion of natural T cell and/or treated and/or untreated effector cells and are present in a patient's blood and/or at a site of treatment in effective concentration of between about 1 fM and about 300 μM and wherein the one or more integrin agonist compounds of Formula I and one or more stem/progenitor cells may be administered separately, collectively, and/or concurrently. In certain embodiments, the one or more integrin agonist compounds of Formula I may be administered before, during and after the administration of the one or more stem/progenitor cells. In certain embodiments, the one or more integrin agonist compounds of Formula I may be administered in an administration schedule of separate doses before administration of the one or more stem/progenitor cells, concurrent with the administration of the one or more stem/progenitor cells, and/or in an administration schedule of separate doses after administration of the one or more stem/progenitor cells, wherein the administration schedules may be the same or different. In other embodiments, the administration schedules may comprise dosing several days before the administration of the one or more stem/progenitor cells and for several day after the administration of the one or more stem/progenitor cells.

### Integrin Agonist and Therapeutic Antibody Treating Compositions

Embodiments of this disclosure include compositions including one or more integrin agonist compounds of Formula I and one or more therapeutic antibodies for treating cancer, especially treating solid tumor, wherein the one or more integrin agonist compounds of Formula I enhance homing, infiltration, engrafting, and invasion of natural T cell and/or treated and/or untreated effector cells and are present in a patient's blood and/or at a site of treatment in effective concentration of between about 1 fM and about 300 μM and wherein the one or more integrin agonist compounds of Formula I and one or more therapeutic antibodies may be administered separately, collectively, and/or concurrently and methods for administering the compositions. In certain embodiments, the one or more integrin agonist compounds of Formula I may be administered before, during and after the administration of the one or more therapeutic antibodies. In certain embodiments, the one or more integrin agonist compounds of Formula I may be administered in an administration schedule of separate doses before administration of the one or more therapeutic antibodies, concurrent with the administration of the one or more therapeutic antibodies, and/or in an administration schedule of separate doses after administration of the one or more therapeutic antibodies, wherein the administration schedules may be the same or different. In other embodiments, the administration schedules may comprise dosing several days before the administration of the one or more therapeutic antibodies and for several day after the administration of the one or more therapeutic antibodies.

### Integrin Agonist and Immunomodulator Treating Compositions

Embodiments of this disclosure include compositions including one or more integrin agonist compounds of Formula I and one or more immunomodulators for treating cancer, especially treating solid tumor, wherein the one or more integrin agonist compounds of Formula I enhance homing, infiltration, engrafting, and invasion of natural T cell and/or treated and/or untreated effector cells and are present in a patient's blood and/or at a site of treatment in effective concentration of between about 1 fM and about 300 μM and wherein the one or more integrin agonist compounds of Formula I and one or more immunomodulators may be administered separately, collectively, and/or concurrently and methods for administering the compositions. In certain embodiments, the one or more integrin agonist compounds of Formula I may be administered before, during and after the administration of the one or more immunomodulators. In certain embodiments, the one or more integrin agonist compounds of Formula I may be administered in an administration schedule of separate doses before administration of the one or more immunomodulators, concurrent with the administration of the one or more immunomodulators, and/or in an administration schedule of separate doses after administration of the one or more immunomodulators, wherein the administration schedules may be the same or different. In other embodiments, the administration schedules may comprise dosing several days before the administration of the one or more immunomodulators and for several day after the administration of the one or more immunomodulators.

### Integrin Agonist and Antigen Treating Compositions

Embodiments of this disclosure include compositions including one or more integrin agonist compounds of Formula I and one or more antigens for treating cancer, especially treating solid tumor, wherein the one or more integrin agonist compounds of Formula I enhance homing, infiltration, engrafting, and invasion of natural T cell and/or treated and/or untreated effector cells and are present in a patient's blood and/or at a site of treatment in effective concentration of between about 1 fM and about 300 μM and wherein the one or more integrin agonist compounds of Formula I and one or more antigens may be administered separately, collectively, and/or concurrently and methods for administering the compositions. In certain embodiments, the one or more integrin agonist compounds of Formula I may be administered before, during and after the administration of the one or more antigens. In certain embodiments, the one or more integrin agonist compounds of Formula I may be administered in an administration schedule of separate doses before administration of the one or more antigens, concurrent with the administration of the one or more antigens, and/or in an administration schedule of separate doses after administration of the one or more antigens, wherein the administration schedules may be the same or different. In other embodiments, the administration schedules may comprise dosing several days before the administration of the one or more antigens and for several day after the administration of the one or more antigens.

### Integrin Agonist and Vaccine Treating Compositions

Embodiments of this disclosure include compositions including one or more integrin agonist compounds of Formula I and one or more vaccines for treating cancer, especially treating solid tumor, wherein the one or more integrin agonist compounds of Formula I enhance homing, infiltration, engrafting, and invasion of natural T cell and/or treated and/or untreated effector cells and are present in a patient's blood and/or at a site of treatment in effective concentration of between about 1 fM and about 300 μM and wherein the one or more integrin agonist compounds of Formula I and one or more vaccines may be administered separately, collectively, and/or concurrently and methods for administering the compositions. In certain embodiments, the one or more integrin agonist compounds of Formula I may be administered before, during and after the administration of the one or more vaccines. In certain embodiments, the one or more integrin agonist compounds of Formula I may be administered in an administration schedule of separate doses before administration of the one or more vaccines, concurrent with the administration of the one or more vaccines, and/or in an administration schedule of separate doses after administration of the one or more vaccines, wherein the administration schedules may be the same or different. In other embodiments, the administration schedules may comprise dosing several days before the administration of the one or more vaccines and for several day after the administration of the one or more vaccines.

### Agonist Delivered Independent of Cells

Agonist can also be delivered independent of the cells for the cell-based therapies described above. In these cases, agonist would be delivered one or more times prior to and/or post cell treatment to promote cell homing, adhesion, and engraftment. Agonist as described herein may also be used to augment treatments unrelated to cell-based therapies, including as an adjuvant for vaccines, and as a treatment of cancer as a monotherapy or in combination with other therapies, including checkpoint blockade antibodies, radiation, or other small molecule anti-cancer drugs.

The compounds and processes described herein will be better understood in connection with the following synthetic schemes which illustrate the methods by which the disclosed compounds may be prepared. A detailed description of the preparation of representative agonist compounds is set forth in the Examples. It should be understood that the same or similar synthetic methods may also be used to synthesize other agonist compounds disclosed herein. These Examples are presented to describe preferred embodiments and uses of the compounds and agonist-treated cells, and are non-limiting in their scope. Immunomodulators target CTLA-4, PD-1, PDL-1, 4-1BB, TIM-1, LAG-3, IDO-1, TIGIT, STING, interleukins, interferons, ionizing radiation, Toll Like Receptors, and further receptors.

Exemplary commercial examples of PD-1 inhibitors include, without limitation, Pembrolizumab (Keytruda), Nivolumab (Opdivo), Cemiplimab (Libtayo), any other PD-1 inhibitor, and mixtures or combinations thereof.

Exemplary commercial examples of PD-L1 inhibitors include, without limitation, Atezolizumab (Tecentriq), Avelumab (Bavencio), Durvalumab (Imfinzi), any other PD-L1 inhibitor, and mixtures or combinations thereof.

Exemplary commercial examples of CTLA-4 inhibitor include, without limitation, Ipilimumab (Yervoy), any other CTLA-4 inhibitor, and mixtures or combinations thereof.

Exemplary commercial examples of Interferons include, without limitation, interferon alpha (Roferon-A, Intron A, Alferon), any other interferon, and mixtures or combinations thereof.

Exemplary examples of Interleukins include, without limitation, interleukin-2 (IL-2) or aldesleukin (Proleukin), and mixtures or combinations thereof.

Exemplary examples of Oncolytic viruses include, without limitation, Talimogene laherparepve (Imlygic), any other Oncolytic virus, and mixtures or combinations thereof.

Exemplary examples of therapeutic antibodies include, without limitation, trastuzumab, cetuximab, ipilimumab, nivolumab rituximab, alemtuzumab, atumumab, tositumomab, any other similar therapeutic antibody, and mixtures or combinations thereof.

Exemplary examples of vaccines include, without limitation, Covid19 vaccines, Anti-Cancer vaccines, Adenovirus vaccines; Anthrax vaccines such as AVA (BioThrax); Cholera vaccines such as Vaxchora; Diphtheria vaccines such as DTaP (Daptacel, Infanrix, Td (Tenivac, generic, DT (-generic-, Tdap (Adacel, Boostrix, DTaP-IPV (Kinrix, Quadracel, DTaP-HepB-IPV (Pediarix, DTaP-IPV/Hib (Pentacel); Hepatitis A vaccines such as HepA (Havrix, Vaqta, HepA-HepB (Twinrix); Hepatitis B vaccines such as HepB (Engerix-B, Recombivax HB, Heplisav-B, DTaP-HepB-IPV (Pediarix, HepA-HepB (Twinrix); Hepatitis C vaccines, Haemophilus influenzae type b (Hib) vaccines such as Hib (ActHIB, PedvaxHIB, Hiberix, DTaP-IPV/Hib (Pentacel); Human Papillomavirus (HPV) vaccines such as HPV9 (Gardasil 9) (For scientific papers, the preferred abbreviation is 9vHPV); seasonal influenza (Flu) only vaccines such as IIV* (Afluria, Fluad, Flublok, Flucelvax, FluLaval, Fluarix, Fluvirin, Fluzone, Fluzone High-Dose, Fluzone Intradermal), LAIV (FluMist), and inactivated flu vaccines such as IIV3, IIV4, RIV3, RIV4 and ccIIV4; Japanese Encephalitis vaccines such as JE (Ixiaro); measles vaccines such as MMR (M-M-R II, MMRV (ProQuad); Meningococcal vaccines such as MenACWY (Menactra, Menveo, MenB (Bexsero, Trumenba); Mumps vaccines such as MMR (M-M-R II, MMRV (ProQuad); Pertussis vaccines such as DTaP (Daptacel, Infanrix, Tdap (Adacel, Boostrix, DTaP-IPV (Kinrix, Quadracel, DTaP-HepB-IPV (Pediarix, DTaP-IPV/Hib (Pentacel); Pneumococcal vaccines such as PCV13 (Prevnar13, PPSV23 (Pneumovax 23); Polio vaccines such as Polio (Ipol, DTaP-IPV (Kinrix, Quadracel, DTaP-HepB-IPV (Pediarix, DTaP-IPV/Hib (Pentacel); Rabies vaccines such as Rabies (Imovax Rabies, RabAvert); Rotavirus vaccines such as RV1 (Rotarix, RV5 (RotaTeq); Rubella vaccines such as MMR (M-M-R II, MMRV (ProQuad); Shingles vaccines such as RZV (Shingrix); Smallpox vaccines such as Vaccinia (ACAM2000); Tetanus vaccines such as DTaP (Daptacel, Infanrix, Td (Tenivac, generic, DT (-generic-, Tdap (Adacel, Boostrix, DTaP-IPV (Kinrix, Quadracel, DTaP-HepB-IPV (Pediarix, DTaP-IPV/Hib (Pentacel); Tuberculosis vaccines, Typhoid Fever vaccines such as Typhoid Oral (Vivotif, Typhoid Polysaccharide (Typhim Vi); Varicella vaccines such as VAR (Varivax, MMRV (ProQuad); and Yellow Fever vaccines such as YF (YF-Vax).

### Additional Disclosure

The following are non-limiting, specific aspects in accordance with the present disclosure;

A first embodiment which is the compound of Formula I,

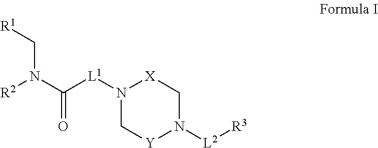

wherein R¹ is an aryl ring; R² comprises an aryl group, an aralkyl group, or a lower alkyl group; L¹ is a linker selected from a group consisting essentially of —(CH₂)ₙ—, —O(CH₂)ₙ—, and —(CH₂)ₙO(CH₂)ₚ—; L² is a linker selected from a group consisting essentially of —CO—, —CO(CH₂)ₘ, —COO(CH₂)ₘ—, —(CH₂)ₘ—, —(CH₂)ₘO—, and —(CH₂)ₘO(CH₂)_{q}—; R³ is selected from a group consisting essentially of aryl, heterocyclyl, CONR⁴R⁵, and —COR⁶; X and Y are independently selected from —CH₂— and —C(O)—; n is an integer of from 1 to 4; m, p, and q, each of which when present, are independently an integer of from 1 to 2; R⁴ and R⁵, when present, are independently selected from a group consisting essentially of hydrogen, a lower alkyl group and an aralkyl group; R⁶, when present, is a heterocyclic ring; when present, each R¹ and R² may be unsubstituted or substituted with a substituent selected from the group consisting essentially of a lower alkyl group, an alkoxy group, a hydroxyalkyl group, —OH, an alkoxyalkyl group, a (C₁-C₃ alkyl)₂amino group, an alkoxyalkoxy group, a cycloalkyl, cycloalkylalkyl group, an aryl group, a heterocyclyl group, an alkylaryl group, an aralkyl group, an alkylheterocyclyl group, and a heterocyclylalkyl group; R³, R⁴, R⁵ and R⁶ may be unsubstituted or substituted with a substituent selected from the group consisting essentially of hydroxy, alkoxy, dialkylamino, halogen, a lower alkyl group, a hydroxyalkyl group, an aliphatic acyl group, —CF₃, oxo, —CN, an alkoxyalkyl group, a (C₁-C₃ alkyl)₂amino group, an alkoxyalkoxy group, a cycloalkyl group, a cycloalkylalkyl group, an aryl group, a heterocyclyl group, an alkylaryl group, an aralkyl group, an alkylheterocyclyl group, a heterocyclylalkyl group, and an aryloxyalkyl group; and pharmaceutically acceptable salts thereof.

A second embodiment which is the compound of the first embodiment wherein R¹ is selected from the group comprising substituted phenyl, and substituted or unsubstituted heteroaromatics selected from: thienyl, oxazolyl, isoxazolyl, pyrrolyl or pyridyl.

A third embodiment which is the compound of any of the first through second embodiments wherein R³ is selected from the group consisting essentially of:

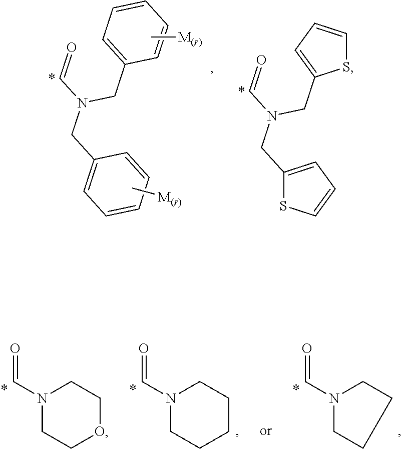

the asterisk * represents the attachment to L²;
wherein each M, when present, is selected from the group consisting essentially of hydroxy, alkoxy, dialkylamino, halogen, and alkyl; and each r, when present, is an integer from 1-2.

A fourth embodiment, which is the compound of any of the first through third embodiments selected from the group consisting essentially of: 2,2′-((piperazine-1,4-diylbis(ethane-2,1-diyl))bis(oxy))bis(N,N-bis(thiophen-2-ylmethyl)acetamide), piperazine-1,4-diylbis(ethane-2,1-diyl) bis(bis(3-methoxybenzyl)carbamate); benzyl 4-(2-((4-(dimethylamino)benzyl)(4-methoxybenzyl)amino)-2-oxoethyl)piperazine-1-carboxylate; 2,2′-(2,5-dioxopiperazine-1,4-diyl)bis(N,N-bis(thiophen-2-ylmethyl)acetamide); benzyl 4-(2-((4-hydroxybenzyl)(4-methoxybenzyl)amino)-2-oxoethyl)piperazine-1-carboxylate; benzyl 4-(2-((4-(dimethylamino)benzyl)(4-hydroxybenzyl)amino)-2-oxoethyl)piperazine-1-carboxylate dihydrochloride; benzyl 4-(2-(bis(4-hydroxybenzyl)amino)-2-oxoethyl)piperazine-1-carboxylate; benzyl 4-(2-(bis(4-methoxybenzyl)amino)-2-oxoethyl)piperazine-1-carboxylate; benzyl 4-(2-(bis(3-methoxybenzyl)amino)-2-oxoethyl)piperazine-1-carboxylate; piperazine-1,4-diylbis(ethane-2,1-diyl) bis(4-(dimethylamino)benzyl (4-methoxybenzyl)carbamate); 2,2′-(2,5-dioxopiperazine-1,4-diyl)bis(N-(3-(dimethylamino)benzyl)-N-(4-methoxybenzyl)acetamide); 2,2′-(2,5-dioxopiperazine-1,4-diyl)bis(N,N-bis(3-methoxybenzyl)acetamide); 2,2′-(piperazine-1,4-diyl)bis(N,N-bis(4-methoxybenzyl)acetamide); benzyl 4-(4-(bis(4-methoxybenzyl)amino)-4-oxobutyl)piperazine-1-carboxylate; benzyl 4-(5-(bis(thiophen-2-ylmethyl)amino)-5-oxopentyl)piperazine-1-carboxylate; 2,2′-(piperazine-1,4-diyl)bis(N,N-bis(thiophen-2-ylmethyl)acetamide); 3-methoxybenzyl 4-(2-(bis(4-methoxybenzyl)amino)-2-oxoethyl)piperazine-1-carboxylate; benzyl 4-(5-(bis(3-methoxybenzyl)amino)-5-oxopentyl)piperazine-1-carboxylate; benzyl 4-(5-(bis(4-methoxybenzyl)amino)-5-oxopentyl)piperazine-1-carboxylate; 4-methoxyphenethyl 4-(2-(bis(4-methoxybenzyl)amino)-2-oxoethyl)piperazine-1-carboxylate; 2,2′-(2-oxopiperazine-1,4-diyl)bis(N,N-bis(4-methoxybenzyl)acetamide); 3-methoxybenzyl 4-(2-((4-(dimethylamino)benzyl)(4-methoxybenzyl)amino)-2-oxoethyl)-3-oxopiperazine-1-carboxylate; N,N-bis(4-methoxybenzyl)-2-(4-(3-methoxybenzyl)-2-oxopiperazin-1-yl)acetamide; N-(4-(dimethylamino)benzyl)-2-(4-(3-methoxybenzoyl)-2-oxopiperazin-1-yl)-N-(4-methoxybenzyl)acetamide; 2-(4-(3-methoxybenzoyl)-2-oxopiperazin-1-yl)-N,N-bis(4-methoxybenzyl)acetamide; N,N-bis(4-methoxybenzyl)-2-(4-(2-(3-methoxyphenyl)acetyl)-2-oxopiperazin-1-yl)acetamide; (2,5-dioxopiperazine-1,4-diyl)bis(ethane-2,1-diyl) bis(bis(3-methoxybenzyl)carbamate); (2,5-dioxopiperazine-1,4-diyl)bis(ethane-2,1-diyl) bis(bis(4-methoxybenzyl)carbamate); (2,5-dioxopiperazine-1,4-diyl)bis(ethane-2,1-diyl) bis(4-(dimethylamino)benzyl (3-methoxybenzyl)carbamate); (2,5-dioxopiperazine-1,4-diyl)bis(ethane-2,1-diyl) bis(4-(dimethylamino)benzyl (4-methoxybenzyl)carbamate); (2,5-dioxopiperazine-1,4-diyl)bis(ethane-2,1-diyl) bis(bis(thiophen-2-ylmethyl)carbamate); 2,2′-(piperazine-1,4-diyl)bis(N-isobutyl-N-(4-methoxybenzyl)acetamide); 2,2′-(2-oxopiperazine-1,4-diyl)bis(N-isobutyl-N-(4-methoxybenzyl)acetamide); 2,2′-(2,5-dioxopiperazine-1,4-diyl)bis(N-isobutyl-N-(4-methoxybenzyl)acetamide); 2,2′-(2-oxopiperazine-1,4-diyl)bis(N-(4-(dimethylamino)benzyl)-N-(4-methoxybenzyl)acetamide); 2,2′-(2,5-dioxopiperazine-1,4-diyl)bis(N,N-bis(4-(dimethylamino)benzyl)acetamide), solvates thereof, precursors thereof and hydrates thereof.

A fifth embodiment which is the compound of any of the first through fourth embodiments wherein the log P is less than about 6,

A sixth embodiment which is a pharmaceutical composition comprising: a compound of Formula I,

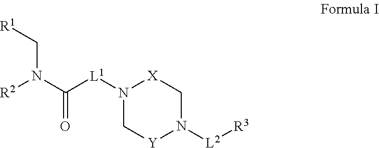

wherein R¹ is an aryl ring, R² comprises an aryl group, an aralkyl group, or a lower alkyl group, L′ is a linker selected from a group consisting essentially of —(CH₂)ₙ—, —O(CH₂)ₙ—, and —(CH₂)ₙO(CH₂)ₚ-L² is a linker selected from a group consisting essentially of —CO—, —CO(CH₂)ₘ, —COO(CH₂)ₘ—, —(CH₂)ₘ—, —(CH₂)ₘO—, and —(CH₂)ₘO(CH₂)_{q}—, R³ is selected from a group consisting essentially of aryl, heterocyclyl, CONR⁴R⁵, and —COR⁶; X and Y are independently selected from —CH₂— and —C(O)—; n is an integer of from 1 to 4; m, p, and q, each of which when present, are independently an integer of from 1 to 2; R⁴ and R⁵, when present, are independently selected from a group consisting essentially of hydrogen, a lower alkyl group and an aralkyl group; R⁶, when present, is a heterocyclic ring; when present, each R¹ and R² may be unsubstituted or substituted with a substituent selected from the group consisting essentially of a lower alkyl group, an alkoxy group, a hydroxyalkyl group, —OH, an alkoxyalkyl group, a (C₁-C₃ alkyl)₂amino group, an alkoxyalkoxy group, a cycloalkyl, cycloalkylalkyl group, an aryl group, a heterocyclyl group, an alkylaryl group, an aralkyl group, an alkylheterocyclyl group, and a heterocyclylalkyl group; R³, R⁴, R⁵ and R⁶ may be unsubstituted or substituted with a substituent selected from the group consisting essentially of hydroxy, alkoxy, dialkylamino, halogen, a lower alkyl group, a hydroxyalkyl group, an aliphatic acyl group, —CF₃, oxo, —CN, an alkoxyalkyl group, a (C₁-C₃ alkyl)₂amino group, an alkoxyalkoxy group, a cycloalkyl group, a cycloalkylalkyl group, an aryl group, a heterocyclyl group, an alkylaryl group, an aralkyl group, an alkylheterocyclyl group, a heterocyclylalkyl group, and an aryloxyalkyl group; and pharmaceutically acceptable salts thereof.

A seventh embodiment which is a medicament for use in the treatment of any condition susceptible of being improved or prevented by the selective occupation of an integrin receptor, comprising the compound of Formula I,

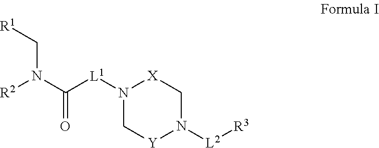

wherein R¹ is an aryl ring, R² comprises an aryl group, an aralkyl group, or a lower alkyl group, L′ is a linker selected from a group consisting essentially of —(CH₂)ₙ—, —O(CH₂)ₙ—, and —(CH₂)ₙO(CH₂)ₚ-L² is a linker selected from a group consisting essentially of —CO—, —CO(CH₂)ₘ, —COO(CH₂)ₘ—, —(CH₂)ₘ—, —(CH₂)ₘO—, and —(CH₂)ₘO(CH₂)_{q}—, R³ is selected from a group consisting essentially of aryl, heterocyclyl, CONR⁴R⁵, and —COR⁶; X and Y are independently selected from —CH₂— and —C(O)—; n is an integer of from 1 to 4; m, p, and q, each of which when present, are independently an integer of from 1 to 2; R⁴ and R⁵, when present, are independently selected from a group consisting essentially of hydrogen, a lower alkyl group and an aralkyl group; R⁶, when present, is a heterocyclic ring; when present, each R¹ and R² may be unsubstituted or substituted with a substituent selected from the group consisting essentially of a lower alkyl group, an alkoxy group, a hydroxyalkyl group, —OH, an alkoxyalkyl group, a (C₁-C₃ alkyl)₂amino group, an alkoxyalkoxy group, a cycloalkyl, cycloalkylalkyl group, an aryl group, a heterocyclyl group, an alkylaryl group, an aralkyl group, an alkylheterocyclyl group, and a heterocyclylalkyl group; R³, R⁴, R⁵ and R⁶ may be unsubstituted or substituted with a substituent selected from the group consisting essentially of hydroxy, alkoxy, dialkylamino, halogen, a lower alkyl group, a hydroxyalkyl group, an aliphatic acyl group, —CF₃, oxo, —CN, an alkoxyalkyl group, a (C₁-C₃ alkyl)₂amino group, an alkoxyalkoxy group, a cycloalkyl group, a cycloalkylalkyl group, an aryl group, a heterocyclyl group, an alkylaryl group, an aralkyl group, an alkylheterocyclyl group, a heterocyclylalkyl group, and an aryloxyalkyl group; and pharmaceutically acceptable salts thereof.

An eighth embodiment which is the medicament of the seventh embodiment wherein the integrin is selected from the group consisting essentially of α4β1, α5β1, α4β7, and αLβ2.

A ninth embodiment which is the medicament of any of the seventh through eighth embodiments, further comprising a pharmaceutically acceptable excipient, a pharmaceutically acceptable carrier or both.

A tenth embodiment which is a liposome comprising a compound selected from the group consisting essentially of: 2,2′-((piperazine-1,4-diylbis(ethane-2,1-diyl))bis(oxy))bis(N,N-bis(thiophen-2-ylmethyl)acetamide), piperazine-1,4-diylbis(ethane-2,1-diyl) bis(bis(3-methoxybenzyl)carbamate); benzyl 4-(2-((4-(dimethylamino)benzyl)(4-methoxybenzyl)amino)-2-oxoethyl)piperazine-1-carboxylate; 2,2′-(2,5-dioxopiperazine-1,4-diyl)bis(N,N-bis(thiophen-2-ylmethyl)acetamide); benzyl 4-(2-((4-hydroxybenzyl)(4-methoxybenzyl)amino)-2-oxoethyl)piperazine-1-carboxylate; benzyl 4-(2-((4-(dimethylamino)benzyl)(4-hydroxybenzyl)amino)-2-oxoethyl)piperazine-1-carboxylate dihydrochloride; benzyl 4-(2-(bis(4-hydroxybenzyl)amino)-2-oxoethyl)piperazine-1-carboxylate; benzyl 4-(2-(bis(4-methoxybenzyl)amino)-2-oxoethyl)piperazine-1-carboxylate; benzyl 4-(2-(bis(3-methoxybenzyl)amino)-2-oxoethyl)piperazine-1-carboxylate; piperazine-1,4-diylbis(ethane-2,1-diyl) bis(4-(dimethylamino)benzyl (4-methoxybenzyl)carbamate); 2,2′-(2,5-dioxopiperazine-1,4-diyl)bis(N-(3-(dimethylamino)benzyl)-N-(4-methoxybenzyl)acetamide); 2,2′-(2,5-dioxopiperazine-1,4-diyl)bis(N,N-bis(3-methoxybenzyl)acetamide); 2,2′-(piperazine-1,4-diyl)bis(N,N-bis(4-methoxybenzyl)acetamide); benzyl 4-(4-(bis(4-methoxybenzyl)amino)-4-oxobutyl)piperazine-1-carboxylate; benzyl 4-(5-(bis(thiophen-2-ylmethyl)amino)-5-oxopentyl)piperazine-1-carboxylate; 2,2′-(piperazine-1,4-diyl)bis(N,N-bis(thiophen-2-ylmethyl)acetamide); 3-methoxybenzyl 4-(2-(bis(4-methoxybenzyl)amino)-2-oxoethyl)piperazine-1-carboxylate; benzyl 4-(5-(bis(3-methoxybenzyl)amino)-5-oxopentyl)piperazine-1-carboxylate; benzyl 4-(5-(bis(4-methoxybenzyl)amino)-5-oxopentyl)piperazine-1-carboxylate; 4-methoxyphenethyl 4-(2-(bis(4-methoxybenzyl)amino)-2-oxoethyl)piperazine-1-carboxylate; 2,2′-(2-oxopiperazine-1,4-diyl)bis(N,N-bis(4-methoxybenzyl)acetamide); 3-methoxybenzyl 4-(2-((4-(dimethylamino)benzyl)(4-methoxybenzyl)amino)-2-oxoethyl)-3-oxopiperazine-1-carboxylate; N,N-bis(4-methoxybenzyl)-2-(4-(3-methoxybenzyl)-2-oxopiperazin-1-yl)acetamide; N-(4-(dimethylamino)benzyl)-2-(4-(3-methoxybenzoyl)-2-oxopiperazin-1-yl)-N-(4-methoxybenzyl)acetamide; 2-(4-(3-methoxybenzoyl)-2-oxopiperazin-1-yl)-N,N-bis(4-methoxybenzyl)acetamide; N,N-bis(4-methoxybenzyl)-2-(4-(2-(3-methoxyphenyl)acetyl)-2-oxopiperazin-1-yl)acetamide; (2,5-dioxopiperazine-1,4-diyl)bis(ethane-2,1-diyl) bis(bis(3-methoxybenzyl)carbamate); (2,5-dioxopiperazine-1,4-diyl)bis(ethane-2,1-diyl) bis(bis(4-methoxybenzyl)carbamate); (2,5-dioxopiperazine-1,4-diyl)bis(ethane-2,1-diyl) bis(4-(dimethylamino)benzyl (3-methoxybenzyl)carbamate); (2,5-dioxopiperazine-1,4-diyl)bis(ethane-2,1-diyl) bis(4-(dimethylamino)benzyl (4-methoxybenzyl)carbamate); (2,5-dioxopiperazine-1,4-diyl)bis(ethane-2,1-diyl) bis(bis(thiophen-2-ylmethyl)carbamate); 2,2′-(piperazine-1,4-diyl)bis(N-isobutyl-N-(4-methoxybenzyl)acetamide); 2,2′-(2-oxopiperazine-1,4-diyl)bis(N-isobutyl-N-(4-methoxybenzyl)acetamide); 2,2′-(2,5-dioxopiperazine-1,4-diyl)bis(N-isobutyl-N-(4-methoxybenzyl)acetamide); 2,2′-(2-oxopiperazine-1,4-diyl)bis(N-(4-(dimethylamino)benzyl)-N-(4-methoxybenzyl)acetamide); 2,2′-(2,5-dioxopiperazine-1,4-diyl)bis(N,N-bis(4-(dimethylamino)benzyl)acetamide), solvates thereof, precursors thereof and hydrates thereof.

An eleventh embodiment which is an ophthalmic formulation comprising a lymphocyte function-associated antigen-1 (LFA-1) agonist comprising the compound of Formula I,

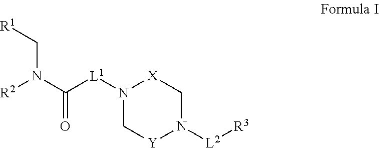

wherein R¹ is an aryl ring, R² comprises an aryl group, an aralkyl group, or a lower alkyl group, L¹ is a linker selected from a group consisting essentially of —(CH₂)ₙ—, —O(CH₂)ₙ—, and —(CH₂)ₙO (CH₂)ₚ-L² is a linker selected from a group consisting essentially of —CO—, —CO(CH₂)ₘ, —COO(CH₂)ₘ—, —(CH₂)ₘ—, —(CH₂)ₘO—, and —(CH₂)ₘO(CH₂)_{q}—, R³ is selected from a group consisting essentially of aryl, heterocyclyl, CONR⁴R⁵, and —COR⁶; X and Y are independently selected from —CH₂— and —C(O)—; n is an integer of from 1 to 4; m, p, and q, each of which when present, are independently an integer of from 1 to 2; R⁴ and R⁵, when present, are independently selected from a group consisting essentially of hydrogen, a lower alkyl group and an aralkyl group; R⁶, when present, is a heterocyclic ring; when present, each R¹ and R² may be unsubstituted or substituted with a substituent selected from the group consisting essentially of a lower alkyl group, an alkoxy group, a hydroxyalkyl group, —OH, an alkoxyalkyl group, a (C₁-C₃ alkyl)₂amino group, an alkoxyalkoxy group, a cycloalkyl, cycloalkylalkyl group, an aryl group, a heterocyclyl group, an alkylaryl group, an aralkyl group, an alkylheterocyclyl group, and a heterocyclylalkyl group; R³, R⁴, R⁵ and R⁶ may be unsubstituted or substituted with a substituent selected from the group consisting essentially of hydroxy, alkoxy, dialkylamino, halogen, a lower alkyl group, a hydroxyalkyl group, an aliphatic acyl group, —CF₃, oxo, —CN, an alkoxyalkyl group, a (C₁-C₃ alkyl)₂amino group, an alkoxyalkoxy group, a cycloalkyl group, a cycloalkylalkyl group, an aryl group, a heterocyclyl group, an alkylaryl group, an aralkyl group, an alkylheterocyclyl group, a heterocyclylalkyl group, and an aryloxyalkyl group; and pharmaceutically acceptable salts thereof.

A twelfth embodiment which is the ophthalmic formulation of the eleventh embodiment wherein R³ is selected from the group consisting essentially of:

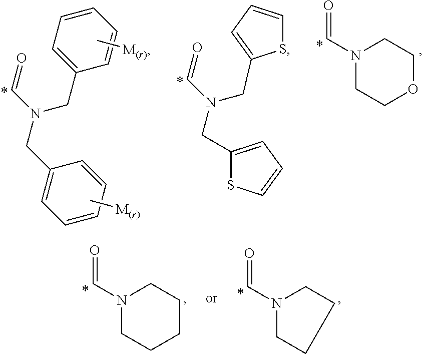

the asterisk * represents the attachment to L²;
   wherein each M, when present, is selected from the groups consisting essentially of hydroxy, alkoxy, dialkylamino, halogen, and alkyl; and each r, when present, is an integer from 1-2.

A thirteenth embodiment which is a complex formed between (i) an integrin expressing cell and a VLA-4 integrin agonist; and (ii) an integrin binding protein wherein the VLA-4 integrin agonist has general Formula I

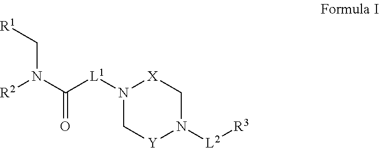

wherein R¹ is an aryl ring, R² comprises an aryl group, an aralkyl group, or a lower alkyl group, L¹ is a linker selected from a group consisting essentially of —(CH₂)ₙ—, —O(CH₂)ₙ—, and —(CH₂)ₙO(CH₂)ₚ-L² is a linker selected from a group consisting essentially of —CO—, —CO(CH₂)ₘ, —COO(CH₂)ₘ—, —(CH₂)ₘ—, —(CH₂)ₘO—, and —(CH₂)ₘO(CH₂)_{q}—, R³ is selected from a group consisting essentially of aryl, heterocyclyl, CONR⁴R⁵, and —COR⁶; X and Y are independently selected from —CH₂— and —C(O)—; n is an integer of from 1 to 4; m, p, and q, each of which when present, are independently an integer of from 1 to 2; R⁴ and R⁵, when present, are independently selected from a group consisting essentially of hydrogen, a lower alkyl group and an aralkyl group; R⁶, when present, is a heterocyclic ring; when present, each R¹ and R² may be unsubstituted or substituted with a substituent selected from the group consisting essentially of a lower alkyl group, an alkoxy group, a hydroxyalkyl group, —OH, an alkoxyalkyl group, a (C₁-C₃ alkyl)₂amino group, an alkoxyalkoxy group, a cycloalkyl, cycloalkylalkyl group, an aryl group, a heterocyclyl group, an alkylaryl group, an aralkyl group, an alkylheterocyclyl group, and a heterocyclylalkyl group; R³, R⁴, R⁵ and R⁶ may be unsubstituted or substituted with a substituent selected from the group consisting essentially of hydroxy, alkoxy, dialkylamino, halogen, a lower alkyl group, a hydroxyalkyl group, an aliphatic acyl group, —CF₃, oxo, —CN, an alkoxyalkyl group, a (C₁-C₃ alkyl)₂amino group, an alkoxyalkoxy group, a cycloalkyl group, a cycloalkylalkyl group, an aryl group, a heterocyclyl group, an alkylaryl group, an aralkyl group, an alkylheterocyclyl group, a heterocyclylalkyl group, and an aryloxyalkyl group; and pharmaceutically acceptable salts thereof.

A fourteenth embodiment which is the complex of the thirteenth embodiment wherein R³ is selected from the group consisting essentially of:

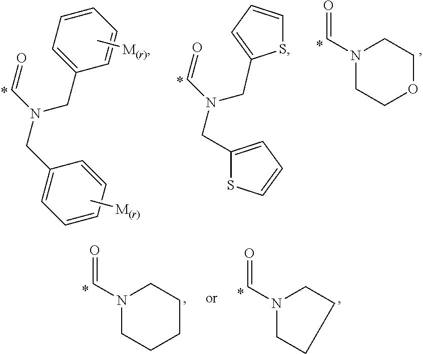

the asterisk * represents the attachment to L²;
   wherein each M, when present, is selected from the groups consisting essentially of hydroxy, alkoxy, dialkylamino, halogen, and alkyl; and each r, when present, is an integer from 1-2.

A fifteenth embodiment which is the complex of any of the twelfth through fourteenth embodiments wherein the integrin binding protein comprises vascular cell adhesion molecule-1 (VCAM 1), fibronectin, mucosal addressin cell adhesion molecule-1 (MAdCAM-1), intercellular adhesion molecule-1 (ICAM-1), intercellular adhesion molecule-2 (ICAM-2) or a combination thereof.

A sixteenth embodiment which is the complex of any of the twelfth through fifteenth embodiments wherein the integrin expressing cells comprise embryonic stem cells, adult stem cells, progenitor cells, induced pluripotent stem cells, or a combination thereof.

A seventeenth embodiment which is a method of enhancing retention of exogenously-introduced cells at an in vivo target site in a mammal generally comprising: treating integrin-expressing cells in vitro with an agonist of integrin to produce agonist-treated cells; and introducing at least a portion the agonist-treated cells to an in vivo target site in a mammal; wherein a greater number of the agonist-treated cells remain at the in vivo target site when compared to the number of cells retained when the same integrin-expressing cells are not treated and introduced to the in vivo target site and wherein the agonist of integrin is a VLA-4 integrin agonist.

An eighteenth embodiment which is the method of the seventeenth embodiment wherein the agonist of integrin is a compound selected from the group consisting essentially of: 2,2′-((piperazine-1,4-diylbis(ethane-2,1-diyl))bis(oxy))bis(N,N-bis(thiophen-2-ylmethyl)acetamide), piperazine-1,4-diylbis(ethane-2,1-diyl)bis(bis(3-methoxybenzyl)carbamate); benzyl 4-(2-((4-(dimethylamino)benzyl)(4-methoxybenzyl)amino)-2-oxoethyl)piperazine-1-carboxylate; 2,2′-(2,5-dioxopiperazine-1,4-diyl)bis(N,N-bis(thiophen-2-ylmethyl)acetamide); benzyl 4-(2-((4-hydroxybenzyl)(4-methoxybenzyl)amino)-2-oxoethyl)piperazine-1-carboxylate; benzyl 4-(2-((4-(dimethylamino)benzyl)(4-hydroxybenzyl)amino)-2-oxoethyl)piperazine-1-carboxylate dihydrochloride; benzyl 4-(2-(bis(4-hydroxybenzyl)amino)-2-oxoethyl)piperazine-1-carboxylate; benzyl 4-(2-(bis(4-methoxybenzyl)amino)-2-oxoethyl)piperazine-1-carboxylate; benzyl 4-(2-(bis(3-methoxybenzyl)amino)-2-oxoethyl)piperazine-1-carboxylate; piperazine-1,4-diylbis(ethane-2,1-diyl) bis(4-(dimethylamino)benzyl (4-methoxybenzyl)carbamate); 2,2′-(2,5-dioxopiperazine-1,4-diyl)bis(N-(3-(dimethylamino)benzyl)-N-(4-methoxybenzyl)acetamide); 2,2′-(2,5-dioxopiperazine-1,4-diyl)bis(N,N-bis(3-methoxybenzyl)acetamide); 2,2′-(piperazine-1,4-diyl)bis(N,N-bis(4-methoxybenzyl)acetamide); benzyl 4-(4-(bis(4-methoxybenzyl)amino)-4-oxobutyl)piperazine-1-carboxylate; benzyl 4-(5-(bis(thiophen-2-ylmethyl)amino)-5-oxopentyl)piperazine-1-carboxylate; 2,2′-(piperazine-1,4-diyl)bis(N,N-bis(thiophen-2-ylmethyl)acetamide); 3-methoxybenzyl 4-(2-(bis(4-methoxybenzyl)amino)-2-oxoethyl)piperazine-1-carboxylate; benzyl 4-(5-(bis(3-methoxybenzyl)amino)-5-oxopentyl)piperazine-1-carboxylate; benzyl 4-(5-(bis(4-methoxybenzyl)amino)-5-oxopentyl)piperazine-1-carboxylate; 4-methoxyphenethyl 4-(2-(bis(4-methoxybenzyl)amino)-2-oxoethyl)piperazine-1-carboxylate; 2,2′-(2-oxopiperazine-1,4-diyl)bis(N,N-bis(4-methoxybenzyl)acetamide); 3-methoxybenzyl 4-(2-((4-(dimethylamino)benzyl)(4-methoxybenzyl)amino)-2-oxoethyl)-3-oxopiperazine-1-carboxylate; N,N-bis(4-methoxybenzyl)-2-(4-(3-methoxybenzyl)-2-oxopiperazin-1-yl)acetamide; N-(4-(dimethylamino)benzyl)-2-(4-(3-methoxybenzoyl)-2-oxopiperazin-1-yl)-N-(4-methoxybenzyl)acetamide; 2-(4-(3-methoxybenzoyl)-2-oxopiperazin-1-yl)-N,N-bis(4-methoxybenzyl)acetamide; N,N-bis(4-methoxybenzyl)-2-(4-(2-(3-methoxyphenyl)acetyl)-2-oxopiperazin-1-yl)acetamide; (2,5-dioxopiperazine-1,4-diyl)bis(ethane-2,1-diyl) bis(bis(3-methoxybenzyl)carbamate); (2,5-dioxopiperazine-1,4-diyl)bis(ethane-2,1-diyl) bis(bis(4-methoxybenzyl)carbamate); (2,5-dioxopiperazine-1,4-diyl)bis(ethane-2,1-diyl) bis(4-(dimethylamino)benzyl (3-methoxybenzyl)carbamate); (2,5-dioxopiperazine-1,4-diyl)bis(ethane-2,1-diyl) bis(4-(dimethylamino)benzyl (4-methoxybenzyl)carbamate); (2,5-dioxopiperazine-1,4-diyl)bis(ethane-2,1-diyl) bis(bis(thiophen-2-ylmethyl)carbamate); 2,2′-(piperazine-1,4-diyl)bis(N-isobutyl-N-(4-methoxybenzyl)acetamide); 2,2′-(2-oxopiperazine-1,4-diyl)bis(N-isobutyl-N-(4-methoxybenzyl)acetamide); 2,2′-(2,5-dioxopiperazine-1,4-diyl)bis(N-isobutyl-N-(4-methoxybenzyl)acetamide); 2,2′-(2-oxopiperazine-1,4-diyl)bis(N-(4-(dimethylamino)benzyl)-N-(4-methoxybenzyl)acetamide); 2,2′-(2,5-dioxopiperazine-1,4-diyl)bis(N,N-bis(4-(dimethylamino)benzyl)acetamide), solvates thereof, precursors thereof and hydrates thereof.

A nineteenth embodiment which is the method of any of the seventeenth through eighteenth embodiments wherein the integrin expressing cells comprise embryonic stem cells, adult stem cells, progenitor cells, induced pluripotent stem cells, or a combination thereof.

A twentieth embodiment which is the method of any of the seventeenth through nineteenth embodiments wherein the treated cells are injected directly or in proximity to a site of damaged vascular tissue, diseased vascular tissue or a combination thereof.

A twenty-first embodiment which is the method of any of the seventeenth through twentieth embodiments wherein the target site comprises vascular cell adhesion molecule-1 (VCAM 1), fibronectin, mucosal address in cell adhesion molecule-1 (MAdCAM-1), intercellular adhesion molecule-1 (ICAM-1), or intercellular adhesion molecule-2 (ICAM-2).

### EXAMPLES

### Adhesion Assays

### Reagents and Cell Lines

For in vitro cell adhesion assays, compounds were dissolved in DMSO to make a series of stock solutions such that a 1:100 dilution in assay buffer would yield the desired final working concentrations in 1% DMSO (vehicle). Human VCAM-1 and ICAM-1 were purchased from R&D Systems (Minneapolis, MN). The Jurkat (VCAM-1 assays) and HSB (ICAM-1 assays) cell lines were obtained from American Type Culture Collection (Manassas, VA) and were maintained in recommended culture media.

### Static Cell Adhesion Assays

Assays were performed as previously described in 1. Vanderslice, P., Woodside, D. G., Caivano, A. R., Decker, E. R., Munsch, C. L., Sherwood, S. J., Lejeune, W. S., Miyamoto, Y. J., McIntyre, B. W., Tilton, R. G., and Dixon, R. A. (2010) Potent in vivo suppression of inflammation by selectively targeting the high affinity conformation of integrin alpha4beta1. Biochem Biophys Res Commun 400, 619-624; and 2. Vanderslice, P., Biediger, R. J., Woodside, D. G., Brown, W. S., Khounlo, S., Warier, N. D., Gundlach, C. W. t., Caivano, A. R., Bornmann, W. G., Maxwell, D. S., McIntyre, B. W., Willerson, J. T., and Dixon, R. A. (2013) Small molecule agonist of very late antigen-4 (VLA-4) integrin induces progenitor cell adhesion. J Biol Chem 288, 19414-19428. VCAM-1 or ICAM-1 ligand in 50 μL of 50 mM Tris-HCl (pH 7.4), 150 mM NaCl, (TBS) was added to wells of a 96-well plate and allowed to coat overnight at 4° C. To maximize the window to evaluate agonist activity, a sub-optimal coating concentration of ligand was used, typically between 0.5 and 5 μg/mL in 50 μL TBS for VCAM-1 and ICAM-1 respectively. This ligand concentration corresponded approximately to that which would yield≤ 5% adhesion as determined by dose-response curves. Briefly, 2×10⁶ cells were labeled for 30 minutes with calcein-AM (Molecular Probes), washed, resuspended in binding buffer, and added to triplicate wells of ligand-coated plates (2×10⁵ cells/well) that had been blocked with 2% BSA. After a 30-minute incubation at 37° C., the plates were washed 3 times with binding buffer, the adherent cells were lysed, and fluorescence was measured on a Tecan Safire² plate reader. The binding buffer was PBS with 1 mM MgCl₂, 50% FBS for α4β1/VCAM-1 assays and PBS with 2 mM MgCl₂, 5 mM EGTA, 50% FBS for αLβ2/ICAM-1 assays. EC₅₀ is defined as the concentration of compound required to achieve 50% of the maximal response.

### Example 1: Synthesis of (2,5-dioxopiperazine-1,4-diyl)bis(ethane-2,1-diyl) bis(bis(thiophen-2-ylmethyl)carbamate)(5)

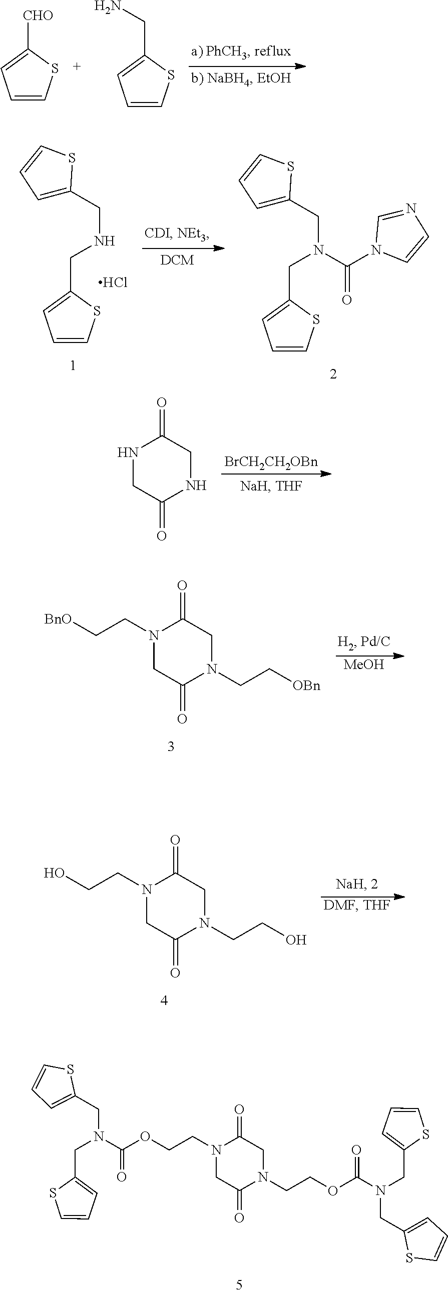

Step One: A solution of thiophene-2-carboxaldehyde (100.1 g, 0.892 mol) and 2-thiophenemethylamine (94.49 g, 0.834 mol) in toluene (300 mL) was refluxed under argon for 3 hours, removing water by means of a Dean-Stark trap. The mixture was cooled to 0° C. in an ice bath, then absolute ethanol (250 mL) was added with stirring, followed by the addition of sodium borohydride (34.79 g, 0.92 mol) was added in six roughly equal portions over the course of 40 minutes. Each portion was added after the foaming subsided from the previous portion. The resulting mixture was allowed to warm to room temperature and stirred overnight, then was cooled to 0° C., and carefully poured into a flask containing aqueous HCl (4M, 500 mL) and ethyl acetate (50 mL) with rapid stirring. A white solid formed and was collected by vacuum filtration, washing with acetone. The white solid was dried under vacuum to give 1 (162.7 g).

This procedure may be used to prepare: bis(3-methoxybenzyl)amine hydrochloride from 3-methoxybenzaldehyde and 3-methoxybenzyl amine; 4-(((4-methoxybenzyl)amino)methyl)-N,N-dimethylaniline dihydrochloride from 4-dimethylaminobenzaldehyde and 4-methoxybenzylamine; 4-(((4-methoxybenzyl)amino)methyl)phenol hydrochloride from 4-hydroxyoxybenzaldehyde and 4-methoxybenzylamine; 4-(((4-(dimethylamino)benzyl)amino)methyl)phenol dihydrochloride from 4-dimethylaminobenzaldehyde and 4-hydroxyoxybenzylamine; 4,4′-(azanediylbis(methylene))diphenol hydrochloride from 4-hydroxybenzaldehyde and 4-hydroxybenzylamine; bis(4-methoxybenzyl)amine hydrochloride (6) from 4-methoxybenzaldehyde and 4-methoxybenzyl amine; and N-(4-methoxybenzyl)-2-methylpropan-1-amine hydrochloride from 4-methoxybenzylamine and isobutyraldehyde.

In some instances, the product was isolated as a freebase by partitioning the hydrochloride salt between dichloromethane or ethyl acetate and an aqueous sodium hydroxide solution. This modification maybe used to prepare: 4-(((4 (dimethylamino)benzyl)amino)methyl)-N,N-dimethylaniline; and 4-(((4-methoxybenzyl)amino)methyl)-N,N-dimethylaniline.

Step Two: To a solution of (1) (46.44 g, 0.189 mol) in dichloromethane (472 mL) at room temperature under argon, triethylamine (31.6 mL, 0.227 mol) was added. The resulting mixture was stirred 15 minutes, then N,N′-carbonyldiimidazole (36.8 g, 0.227 mmol) was added in two portions spaced 10 minutes apart. The resulting mixture was stirred 6 hours, then was diluted with ethyl acetate (800 L) and washed with water (twice) and brine. The organic layer was dried over MgSO₄, then filtered through a 1-inch pad of silica gel, washing with ethyl acetate (500 mL). The filtrate was concentrated under reduced pressure. The residue was taken up in hot ethyl acetate (120 mL) and hexanes (225 mL) was added dropwise from an addition funnel with stirring and intermittent heating. Upon completion of the addition of hexanes, the reaction was stirred while gradually cooling. After stirring overnight, the mixture was filtered, washing with 3:1 hexanes:ethyl acetate, and dried under vacuum to give (2) (46.02) as white crystals.

This procedure may also be used to prepare: N,N-bis(3-methoxybenzyl)-1H-imidazole-1-carboxamide from bis(3-methoxybenzyl)amine hydrochloride; N-(4-(dimethylamino)benzyl)-N-(4-methoxybenzyl)-1H-imidazole-1-carboxamide from 4-(((4-methoxybenzyl)amino)methyl)-N,N-dimethylaniline; N-(4-(dimethylamino)benzyl)-N-(3-methoxybenzyl)-1H-imidazole-1-carboxamide from 4-(((3-methoxybenzyl)amino)methyl)-N,N-dimethylaniline hydrochloride; tert-butyl 4-(1H-imidazole-1-carbonyl)piperazine-1-carboxylate from tert-butyl 4-(1H-imidazole-1-carbonyl)piperazine-1-carboxylate; N,N-bis(4-methoxybenzyl)-1H-imidazole-1-carboxamide from (6); and 4-(1H-imidazole-1-carbonyl)piperazin-2-one from piperazine-2-one.

Step Three: To a suspension of piperazine-2,5-dione (1.00 g, 8.76 mmol) in DMF (30 mL) at room temperature under argon, ((2-bromoethoxy)methyl)benzene (4.1 g, 19.1 mmol) was added. The mixture was stirred for 5 minutes, then sodium hydride (60% dispersion in mineral oil, 770 mg, 19.3 mmol) was added. The mixture was stirred overnight, then the DMF was removed under vacuum. The residue was partitioned between dichloromethane and water. The organic layer was washed with water (5 times) and brine, dried over sodium sulfate, filtered, and concentrated. To the resulting oil, hot hexanes (75 mL) was added. The mixture was heated briefly with swirling, then the hexanes was decanted. This process was repeated, and the remaining oil was dried under reduced pressure to give (3) (2.48 g).

This procedure may also be used to alkylate alcohols. In some cases, the sodium hydride served as a base to consume the hydrogen bromide formed when during alkylation of one or more primary or secondary amines. In some cases, it served to deprotonate an amide and to consume hydrogen bromide formed upon alkylation of an amine. This procedure was also modified to prepare compounds where only one site was alkylated. This procedure or one of these modifications may be used to prepare: 2,2′-((piperazine-1,4-diylbis(ethane-2,1-diyl))bis(oxy))bis(N,N-bis(thiophen-2-ylmethyl)acetamide) [MS (m/z): 337.20 (M+2H)²⁺] from 2-bromo-N,N-bis(thiophen-2-ylmethyl)acetamide and 1,4-bis(2-hydroxyethyl)piperazine; 2,2′-(2,5-dioxopiperazine-1,4-diyl)bis(N,N-bis(thiophen-2-ylmethyl)acetamide) [MS (m/z): 613.24 (M+H)⁺] from glycine anhydride and 2-bromo-N,N-bis(thiophen-2-ylmethyl)acetamide; 2,2′-(2,5-dioxopiperazine-1,4-diyl)bis(N-(3-(dimethylamino)benzyl)-N-(4-methoxybenzyl)acetamide) [MS (m/z): 368.49 (M+2H) 2+] from glycine anhydride and 2-bromo-N-(4-(dimethylamino)benzyl)-N-(4-methoxybenzyl)acetamide; 2,2′-(2,5-dioxopiperazine-1,4-diyl)bis(N,N-bis(3-methoxybenzyl)acetamide) [MS (m/z): 709.41 (M+H)⁺] from glycine anhydride and 2-bromo-N,N-bis(3-methoxybenzyl)acetamide; 3-methoxybenzyl 4-(2-(bis(4-methoxybenzyl)amino)-2-oxoethyl)piperazine-1-carboxylate [MS (m/z): 548.38 (M+H)⁺] from 3-methoxybenzyl piperazine-1-carboxylate hydrochloride and 7; 2,2′-(2-oxopiperazine-1,4-diyl)bis(N,N-bis(4-methoxybenzyl)acetamide) [MS (m/z): 695.47 (M+H)⁺] from 2-bromo-N,N-bis(4-methoxybenzyl)acetamide and N,N-bis(4-methoxybenzyl)-2-(3-oxopiperazin-1-yl)acetamide; 3-methoxybenzyl 4-(2-((4-(dimethylamino)benzyl)(4-methoxybenzyl)amino)-2-oxoethyl)-3-oxopiperazine-1-carboxylate [MS (m/z): 575.29 (M+H)⁺] from 2-bromo-N-(4-(dimethylamino)benzyl)-N-(4-methoxybenzyl)acetamide and 3-methoxybenzyl 3-oxopiperazine-1-carboxylate; N,N-bis(4-methoxybenzyl)-2-(4-(3-methoxybenzyl)-2-oxopiperazin-1-yl)acetamide [MS (m/z): 518.37 (M+H)⁺] from 2-bromo-N,N-bis(4-methoxybenzyl)acetamide and 4-(3-methoxybenzyl)piperazin-2-one; N-(4-(dimethylamino)benzyl)-2-(4-(3-methoxybenzoyl)-2-oxopiperazin-1-yl)-N-(4-methoxybenzyl)acetamide [MS (m/z): 545.34 (M+H)⁺] from 2-bromo-N-(4-(dimethylamino)benzyl)-N-(4-methoxybenzyl)acetamide and 4-(3-methoxybenzoyl)piperazin-2-one; 2-(4-(3-methoxybenzoyl)-2-oxopiperazin-1-yl)-N,N-bis(4-methoxybenzyl)acetamide [MS (m/z): 532.29 (M+H)⁺] from 4-(3-methoxybenzoyl)piperazin-2-one and 2-bromo-N,N-bis(4-methoxybenzyl)acetamide; 2,2′-(piperazine-1,4-diyl)bis(N-isobutyl-N-(4-methoxybenzyl)acetamide) [MS (m/z): 553.47 (M+H)⁺] from 2-bromo-N-isobutyl-N-(4-methoxybenzyl)acetamide and piperazine; 2,2′-(2-oxopiperazine-1,4-diyl)bis(N-isobutyl-N-(4-methoxybenzyl)acetamide) [MS (m/z): 567.45 (M+H)⁺] from piperazine-2-one and 2-bromo-N-isobutyl-N-(4-methoxybenzyl)acetamide; 2,2′-(2,5-dioxopiperazine-1,4-diyl)bis(N-isobutyl-N-(4-methoxybenzyl)acetamide) [MS (m/z): 581.46 (M+H)⁺] from glycine anhydride and 2-bromo-N-isobutyl-N-(4-methoxybenzyl)acetamide; 2,2′-(2-oxopiperazine-1,4-diyl)bis(N-(4-(dimethylamino)benzyl)-N-(4-methoxybenzyl)acetamide) [MS (m/z): 259.32 (M+3H) 3+] from piperazine-2-one and 2-bromo-N-(4-(dimethylamino)benzyl)-N-(4-methoxybenzyl)acetamide; 2,2′-(2,5-dioxopiperazine-1,4-diyl)bis(N,N-bis(4-(dimethylamino)benzyl)acetamide) [MS (m/z): 259.32 (M+3H) 3+] from glycine anhydride and 2-bromo-N,N-bis(4-(dimethylamino)benzyl)acetamide; and N,N-bis(4-methoxybenzyl)-2-(4-(2-(3-methoxyphenyl)acetyl)-2-oxopiperazin-1-yl)acetamide [MS (m/z): 546.36 (M+H)⁺] from 2-bromo-N,N-bis(4-methoxybenzyl)acetamide and 4-(2-(3-methoxyphenyl)acetyl)piperazin-2-one.

Step Four: To a solution of (3) (2.48 g) in anhydrous methanol (50 mL) at room temperature under argon, palladium on carbon (Degussa type E101 NE/W, 10% Pd dry weight basis, 50% water, 700 mg) was added. The atmosphere was exchanged for hydrogen from a balloon (toggle between vacuum and hydrogen several times) and the mixture was stirred overnight. The mixture was filtered through Celite® and the filtrate was concentrated to give (4) (810 mg) as a white solid.

This procedure may also be used to prepare: 2,2′-(2,5-dioxopiperazine-1,4-diyl)diacetic acid from dibenzyl 2,2′-(2,5-dioxopiperazine-1,4-diyl)diacetate.

Step Five: To a mixture of (4) (83 mg, 0.41 mmol) and (2) (374 mg, 1.23 mmol) in DMF (3 mL) and tetrahydrofuran (5 mL) at room temperature under argon, sodium hydride (60% dispersion in mineral oil, 51 mg, 1.28 mmol) was added. The resulting mixture was stirred at room temperature for three days, then was concentrated under reduced pressure. The residue was taken up in dichloromethane, washed with water:brine (4:1, 5 times) and brine, dried over sodium sulfate, filtered and concentrated. The residue was purified by silica gel chromatography, eluting with acetone in hexanes to give (5) [170 mg; MS (m/z): 673.26 (M+H)⁺] as a white solid.

In some instances, a single carbamate group was formed from mono-functional alcohols. This procedure may also be used to prepare: piperazine-1,4-diylbis(ethane-2,1-diyl) bis(bis(3-methoxybenzyl)carbamate) [MS (m/z): 741.53 (M+H)⁺] from 2,2′-(piperazine-1,4-diyl) diethanol and N,N-bis(3-methoxybenzyl)-1H-imidazole-1-carboxamide; piperazine-1,4-diylbis(ethane-2,1-diyl) bis(4-(dimethylamino)benzyl (4-methoxybenzyl)carbamate) [MS (m/z): 384.50 (M+2H) 2+] from 2,2′-(piperazine-1,4-diyl) diethanol and N-(4-(dimethylamino)benzyl)-N-(4-methoxybenzyl)-1H-imidazole-1-carboxamide; 1-tert-butyl 4-(3-methoxybenzyl)piperazine-1,4-dicarboxylate from tert-butyl 4-(1H-imidazole-1-carbonyl)piperazine-1-carboxylate and 3-methoxybenzyl alcohol; 1-tert-butyl 4-(4-methoxyphenethyl)piperazine-1,4-dicarboxylate (8) from 4-methoxyphenethyl alcohol and tert-butyl 4-(1H-imidazole-1-carbonyl)piperazine-1-carboxylate; 3-methoxybenzyl 3-oxopiperazine-1-carboxylate from 4-(1H-imidazole-1-carbonyl)piperazin-2-one and 3-methoxybenzyl alcohol; (2,5-dioxopiperazine-1,4-diyl)bis(ethane-2,1-diyl) bis(bis(3-methoxybenzyl)carbamate) [MS (m/z): 769.40 (M+H)⁺] from 1,4-bis(2-hydroxyethyl)piperazine-2,5-dione and N,N-bis(3-methoxybenzyl)-1H-imidazole-1-carboxamide; (2,5-dioxopiperazine-1,4-diyl)bis(ethane-2,1-diyl) bis(bis(4-methoxybenzyl)carbamate) [MS (m/z): 769.40 (M+H)⁺] from N,N-bis(4-methoxybenzyl)-1H-imidazole-1-carboxamide and 1,4-bis(2-hydroxyethyl)piperazine-2,5-dione; (2,5-dioxopiperazine-1,4-diyl)bis(ethane-2,1-diyl)bis(4-(dimethylamino)benzyl (3-methoxybenzyl)carbamate) [MS (m/z): 398.46 (M+2H) 2+] from 1,4-bis(2-hydroxyethyl)piperazine-2,5-dione and N-(4-(dimethylamino)benzyl)-N-(3-methoxybenzyl)-1H-imidazole-1-carboxamide; and (2,5-dioxopiperazine-1,4-diyl)bis(ethane-2,1-diyl) bis(4-(dimethylamino)benzyl (4-methoxybenzyl)carbamate) [MS (m/z): 398.46 (M+2H) 2+] from 1,4-bis(2-hydroxyethyl)piperazine-2,5-dione and N-(4-(dimethylamino)benzyl)-N-(4-methoxybenzyl)-1H-imidazole-1-carboxamide.

### Example 2: Synthesis of 4-methoxyphenethyl 4-(2-(bis(4-methoxybenzyl)amino)-2-oxoethyl)piperazine-1-carboxylate (10)

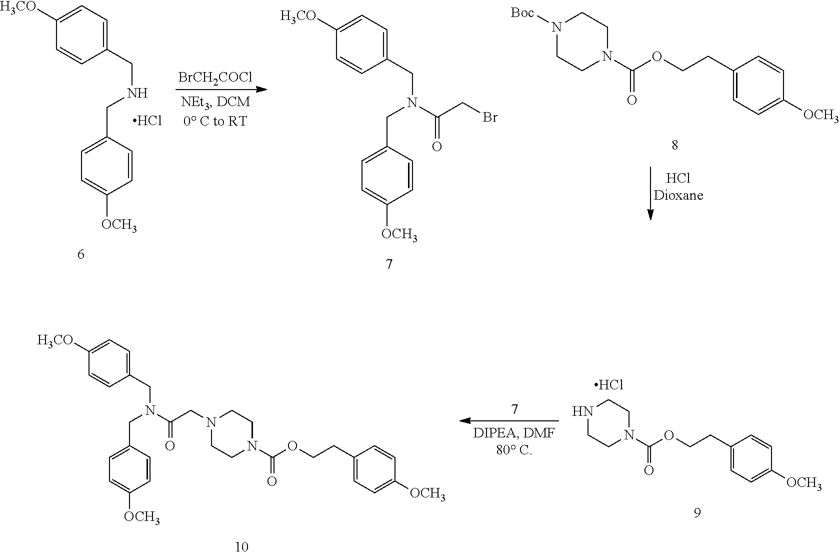

Step One: To a mixture of bis(4-methoxybenzyl)amine hydrochloride (6, 1.00 g, 3.89 mmol) in dichloromethane (15.6 mL) and N,N-disopropylethylamine (1.49 mL, 4.3 mmol) cooled to 0° C. under argon, bromoacetylchloride (0.36 mL, 4.3 mmol) was added dropwise by syringe. The resulting mixture was allowed to gradually warm to room temperature and was stirred overnight. The mixture was washed with aqueous HCl (2N, twice), water and brine, dried over sodium sulfate, filtered and concentrated. The residue was purified by silica gel column to give (7) (634 mg).

This procedure may also be used to prepare: 2-bromo-N,N-bis(thiophen-2-ylmethyl)acetamide from (1); 2-bromo-N,N-bis(3-methoxybenzyl)acetamide from bis(3-methoxybenzyl)amine hydrochloride; 2-bromo-N-(4-(dimethylamino)benzyl)-N-(4-methoxybenzyl)acetamide from 4-(((4-methoxybenzyl)amino)methyl)-N,N-dimethylaniline dihydrochloride; 2-bromo-N-isobutyl-N-(4-methoxybenzyl)acetamide from N-(4-methoxybenzyl)-2-methylpropan-1-amine hydrochloride; and 2-bromo-N,N-bis(4-(dimethylamino)benzyl)acetamide from 4-(((4-(dimethylamino)benzyl)amino)methyl)-N,N-dimethylaniline.

Step Two: To a flask containing (8) (480 mg, 1.32 mmol) at room temperature under argon, HCl in dioxane (4.0 M, 13.2 mmol) was added. The flask was swirled until all the starting material dissolved, then was stirred overnight. The excess HCl was blown off under a stream of argon, then the mixture was concentrated under reduced pressure to give (9) (403 mg).

This procedure may also be used to prepare 3-methoxybenzyl piperazine-1-carboxylate hydrochloride from 1-tert-butyl 4-(3-methoxybenzyl)piperazine-1,4-dicarboxylate.

Step Three: To mixture of (7) (200 mg, 0.53 mmol) and (9) (160 mg, 0.53 mmol) in DMF (2.12 mL) at room temperature under argon, N,N-disopropylethylamine (0.14 mL, 0.80 mmol) was added. The mixture was heated to 80° C. overnight, then was cooled to room temperature, diluted with ethyl acetate, and washed with water (several times) and brine. The organic layer was dried, filtered, and concentrated, and the residue was purified by silica gel chromatography to give (10) [25 mg; MS (m/z): 562.40 (M+H)⁺].

This procedure may also be used to prepare: benzyl 4-(2-ethoxy-2-oxoethyl)piperazine-1-carboxylate from benzyl piperazine-1-carboxylate and ethyl bromoacetate; benzyl 4-(4-methoxy-4-oxobutyl)piperazine-1-carboxylate from 1-Z-piperazine and methyl 4-bromobutyrate; benzyl 4-(5-methoxy-5-oxopentyl)piperazine-1-carboxylate (11) from 1-Z-piperazine and methyl 5-bromovalearate; 4-(3-methoxybenzyl)piperazin-2-one from 3-methoxybenzyl bromide and piperazine-2-one; and N,N-bis(4-methoxybenzyl)-2-(3-oxopiperazin-1-yl)acetamide form 2-bromo-N,N-bis(4-methoxybenzyl)acetamide and piperazine-2-one.

### Example 3: Synthesis of benzyl 4-(5-(bis(thiophen-2-ylmethyl)amino)-5-oxopentyl)piperazine-1-carboxylate (13)

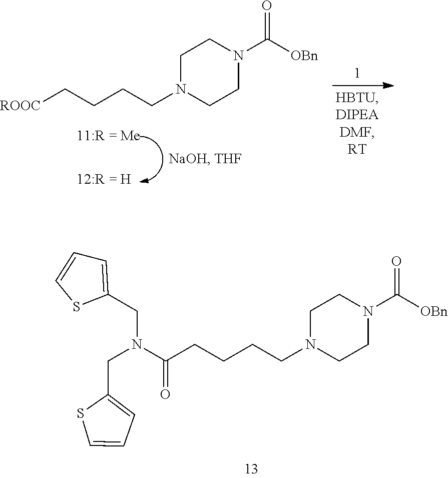

Step One: To a solution of (11) (640 mg, 2.0 mmol) in methanol (2 mL) at room temperature, aqueous sodium hydroxide (2 N, 3 mL, 6 mmol) was added. The mixture was stirred overnight, then was diluted with water and the pH was adjusted to 4-5 with HCl (2N). The mixture was extracted with twice ethyl acetate, the organic phases were combined and washed with brine, dried over sodium sulfate, filtered and concentrated under reduced pressure to give (12) (410 mg).

This procedure may also be used to prepare: 2-(4-((benzyloxy)carbonyl)piperazin-1-yl)acetic acid from benzyl 4-(2-ethoxy-2-oxoethyl)piperazine-1-carboxylate; and 4-(4-((benzyloxy)carbonyl)piperazin-1-yl)butanoic acid from benzyl 4-(4-methoxy-4-oxobutyl)piperazine-1-carboxylate.

Step Two: To a solution of (12) (50 mg, 0.15 mmol) and 1 (41.8 mg, 0.17 mmol) in DMF (0.6 mL) and DIPEA (0.20 mL, 1.1 mmol) at room temperature under argon, HBTU (64.5 mg, 0.17 mmol) was added. The mixture was heated to 50° C. and stirred overnight, then cooled to room temperature, and diluted with water. The mixture was extracted with ethyl acetate, and the organic layer was washed with water and brine, dried over sodium sulfate, filtered, and concentrated under reduced pressure. The residue was purified by silica gel chromatography (Biotage, SNAP10, KP-sil, eluting with 50-100% ethyl acetate in hexanes, then 0-25% methanol in ethyl acetate) to give (13) [23 mg; MS (m/z): 512.32 (M+H)⁺].

In some cases, revers phase chromatography was used to purify the product, which was isolated as a mono- or dihydrochloride salt. This procedure comprising this modification may also be used to prepare: benzyl 4-(2-((4-(dimethylamino)benzyl)(4-methoxybenzyl)amino)-2-oxoethyl)piperazine-1-carboxylate [NMR (300 MHz, DMSO-d₆ {shows to rotational isomers}): δ 7.28-7.42 (m, 5H), 7.07-7.16 (two d, 2H), 6.96-7.06 (two d, 2H), 6.86-6.95 (two d, 2H), 6.73-6.75 (two d, 2H), 5.07 (s, 2H), 4.42 and 4.39 (two s, 2H), 4.31 and 4.27 (two s, 2H), 3.74 and 3.73 (two s, 3H). 3.37 (broad m, partially overlapping H₂O, 4H), 3.25 and 3.20 (two s, 2H), 2.45 (broad m, 4H)] from 4-(((4-methoxybenzyl)amino)methyl)-N,N-dimethylaniline dihydrochloride and 2-(4-((benzyloxy)carbonyl)piperazin-1-yl)acetic acid; benzyl 4-(2-((4-hydroxybenzyl)(4-methoxybenzyl)amino)-2-oxoethyl)piperazine-1-carboxylate [MS (m/z): 504.34 (M+H)⁺] from 4-(((4-methoxybenzyl)amino)methyl)phenol hydrochloride and 2-(4-((benzyloxy)carbonyl)piperazin-1-yl)acetic acid; benzyl 4-(2-((4-(dimethylamino)benzyl)(4-hydroxybenzyl)amino)-2-oxoethyl)piperazine-1-carboxylate dihydrochloride [MS (m/z): 259.32 (M+2H)²⁺] from 4-(((4-(dimethylamino)benzyl)amino)methyl)phenol dihydrochloride and 2-(4-((benzyloxy)carbonyl)piperazin-1-yl)acetic acid; benzyl 4-(2-(bis(4-hydroxybenzyl)amino)-2-oxoethyl)piperazine-1-carboxylate [MS (m/z): 490.36 (M+H)⁺] from 4,4′-(azanediylbis(methylene))diphenol hydrochloride and 2-(4-((benzyloxy)carbonyl)piperazin-1-yl)acetic acid; benzyl 4-(2-(bis(4-methoxybenzyl)amino)-2-oxoethyl)piperazine-1-carboxylate [MS (m/z): 518.37 (M+H)⁺] from (6) and 2-(4-((benzyloxy)carbonyl)piperazin-1-yl)acetic acid; benzyl 4-(2-(bis(3-methoxybenzyl)amino)-2-oxoethyl)piperazine-1-carboxylate [MS (m/z): 518.40 (M+H)⁺] from bis(3-methoxybenzyl)amine hydrochloride and 2-(4-((benzyloxy)carbonyl)piperazin-1-yl)acetic acid; benzyl 4-(4-(bis(4-methoxybenzyl)amino)-4-oxobutyl)piperazine-1-carboxylate [MS (m/z): 546.41 (M+H)⁺] from (6) and 4-(4-((benzyloxy)carbonyl)piperazin-1-yl)butanoic acid; benzyl 4-(5-(bis(3-methoxybenzyl)amino)-5-oxopentyl)piperazine-1-carboxylate [MS (m/z): 560.31 (M+H)⁺] from (12) and bis(3-methoxybenzyl)amine hydrochloride; and benzyl 4-(5-(bis(4-methoxybenzyl)amino)-5-oxopentyl)piperazine-1-carboxylate [MS (m/z): 560.41 (M+H)⁺] from (6) and 5-(4-((benzyloxy)carbonyl)piperazin-1-yl)pentanoic acid.

In some embodiments, an intermediate with two carboxylic acid functional groups was used as a starting material, to prepare diamides, using 2.2 equivalents of each of the amine hydrochloride and HBTU. This modification maybe used to prepare: 2,2′-(piperazine-1,4-diyl)bis(N,N-bis(4-methoxybenzyl)acetamide) [MS (m/z): 681.52 (M+H)⁺] from 2,2′-(piperazine-1,4-diyl)diacetic acid dihydrochloride and bis(4-methoxybenzyl)amine hydrochloride; and 2,2′-(piperazine-1,4-diyl)bis(N,N-bis(thiophen-2-ylmethyl)acetamide) [MS (m/z): 585.30 (M+H)⁺] from (1) and 2,2′-(piperazine-1,4-diyl)diacetic acid dihydrochloride.

### Example 4: Synthesis of 4-(3-methoxybenzoyl)piperazin-2-one (14)

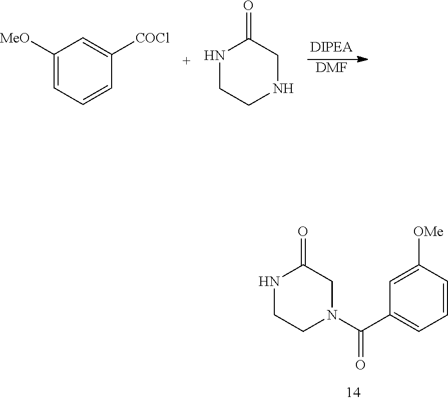

Step One: To a mixture of piperazine-2-one (500 mg, 5.0 mmol) in DMF (2 mL) at room temperature under argon, DIPEA (1.13 mL, 6.5 mmol) and 3-methoxybenzoyl chloride (938 mg, 5.5 mmol) were added sequentially. The mixture was stirred overnight, then was diluted with water and extracted twice with ethyl acetate. The organic phases were combined and washed with brine, dried over sodium sulfate, filtered and concentrated under reduced pressure to give (14)(1.93 g) as a yellow solid.

This procedure was also to prepare 4-(2-(3-methoxyphenyl)acetyl)piperazin-2-one from piperazine-2-one and 2-(3-methoxyphenyl)acetyl chloride.

Lipinski's rule of five (Lipinski, A., Drug Discovery Today: Technologies, Volume 1, Issue 4, 2004, Pages 337-341) discloses the advantages of molecular weights less than 500 and Log Ps less than five in order to effect greater possibility of oral availability and optimal water solubility. Other published compounds acting as VLA-4 integrin agonists have had less than desirable properties in this regard.

| | | | |
|---|---|---|---|
| THI0019 (33 μM against VCAM) | 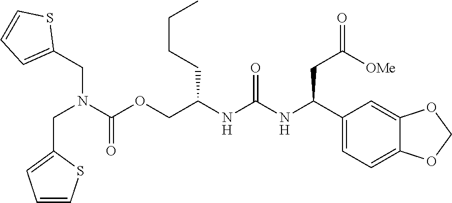 [SMILES: CCCC[C@@H](COC(=O)N(Cc1cccs1)Cc1cccs1)NC(=O)N[C@@H](CC(=O)OC)c1ccc2c(c1)OCO2] | LogP 5.42 | MW 601.73 |
| THI0349 | 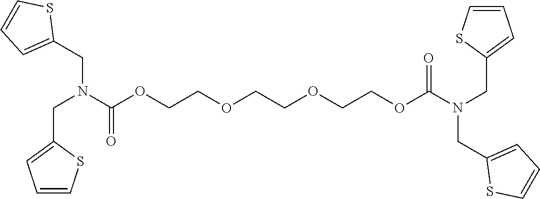 [SMILES: O=C(OCCOCCOCCOC(=O)N(Cc1cccs1)Cc1cccs1)N(Cc1cccs1)Cc1cccs1] | Log P: 6.5 CLogP: 6.4248 | MW 620.82 |

THI0019 has reasonable Log P, but has significantly higher molecular weight than dictated by Lipinski's rules. THI0349, currently in clinical development, likewise has a molecular weight in excess of 600 and Log P of 6.5 or greater. THI0019 and THI0349 (average 11 μM) reported values here were obtained under the assay conditions reported herein and ran higher in the presence of serum than those values under different assay conditions. The following compounds illustrate the class of compounds disclosed herein having properties similar to those suggested by Lipinski's rule of five without sacrificing potency. Relative to THI0019 (average 33 μM) as a control against CS1, the ratio of (compound activity/control) is as follows: Group I< or =1. Group II at least a ratio of >1 up to 2× that of THI0019 ran on the same plate; Group III is >2× of THI0019 but less than 5×; and Group IV>5× of THI0019 (with an upper limit of approximately 200 mM). In relation to LAD (Leukocyte Adhesion Deficiency), LFA-1 ratios are determined by dividing the relative fluorescence units (RFU) of each compound over that of the reference standard of THI0349, (RFU Compound/RFU of THI0349). Against LFA-1, the signal for THI0349 at 3 μM was compared to the claimed compounds at the same concentration. The increased raw response (fluorescence units) demonstrated a more potent agonist against LFA-1 and the ratio would be greater than one (1.0):

### List of Compounds

| | | Average | LAD |
|---|---|---|---|
| | | Ratio | Raw Fluorescence |
| | | Compound | Units against |
| | | to THI0019 | LFA-1; Dose 3 μM; |
| | | (Activity | Ratio of raw |
| | LogP/ | against | counts Compound/ |
| Structure | CLogP | CS1) | THI0349 |
| 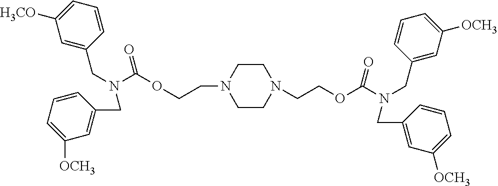 [SMILES: COc1cccc(CN(Cc2cccc(OC)c2)C(=O)OCCN2CCN(CCOC(=O)N(Cc3cccc(OC)c3)Cc3cccc(OC)c3)CC2)c1] | Log P: 7.00 ** | Group I | 4.7 fold increased response |
| 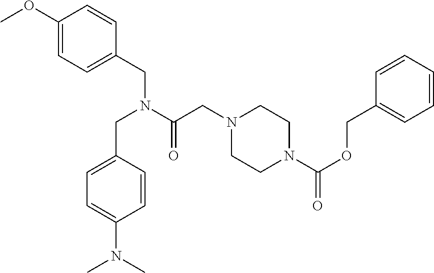 [SMILES: COc1ccc(CN(Cc2ccc(N(C)C)cc2)C(=O)CN2CCN(C(=O)OCc3ccccc3)CC2)cc1] | Log P: 4.3, CLogP: 6.1898 | Group III | 1.2 fold increased response |
| 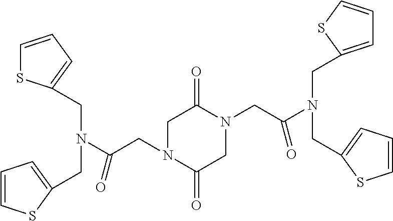 [SMILES: O=C1CN(CC(=O)N(Cc2cccs2)Cc2cccs2)C(=O)CN1CC(=O)N(Cc1cccs1)Cc1cccs1] | Log P: 3.21, CLogP: 4.7356 | Group III | Not assayed |
| 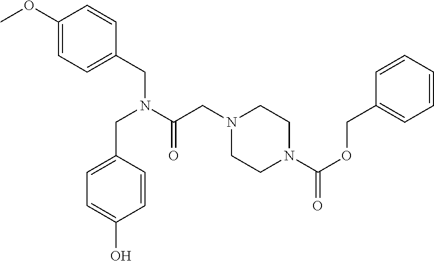 [SMILES: COc1ccc(CN(Cc2ccc(O)cc2)C(=O)CN2CCN(C(=O)OCc3ccccc3)CC2)cc1] | Log P: 3.62, CLogP: 5.3578 | Group II | 1.3 fold increased response |
| 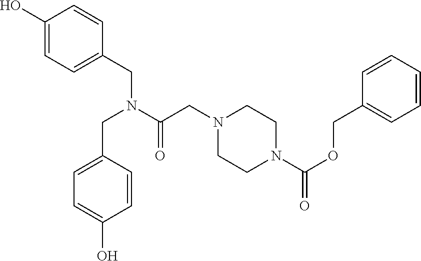 [SMILES: O=C(CN1CCN(C(=O)OCc2ccccc2)CC1)N(Cc1ccc(O)cc1)Cc1ccc(O)cc1] | Log P: 3.36, CLogP: 4.7718 | Group I | 0.3 fold response |
| 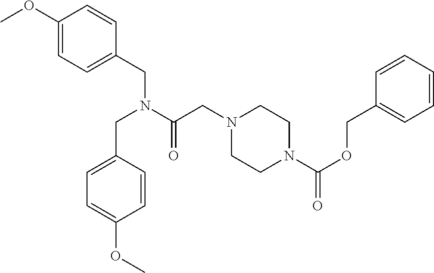 [SMILES: COc1ccc(CN(Cc2ccc(OC)cc2)C(=O)CN2CCN(C(=O)OCc3ccccc3)CC2)cc1] | Log P: 3.89, CLogP: 5.9438 | Group II | 3.3 fold increased response |
| 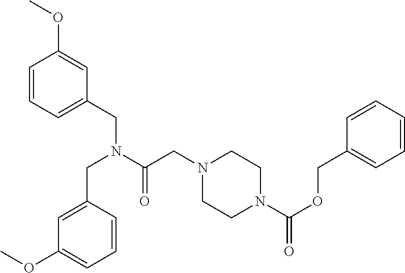 [SMILES: COc1cccc(CN(Cc2cccc(OC)c2)C(=O)CN2CCN(C(=O)OCc3ccccc3)CC2)c1] | Log P: 3.89, CLogP: 5.9438 | Group I | 1.42 fold increased response |
| 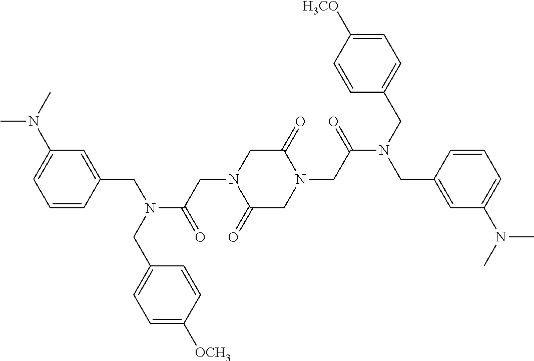 | Log P: 4.41** | Group II | Not assayed |
| 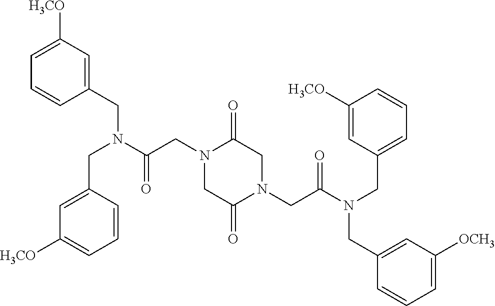 [SMILES: COc1cccc(CN(Cc2cccc(OC)c2)C(=O)CN2CC(=O)N(CC(=O)N(Cc3cccc(OC)c3)Cc3cccc(OC)c3)CC2=O)c1] | Log P: 2.77, CLogP: 5.8936 | Group I | Not Assayed |
| 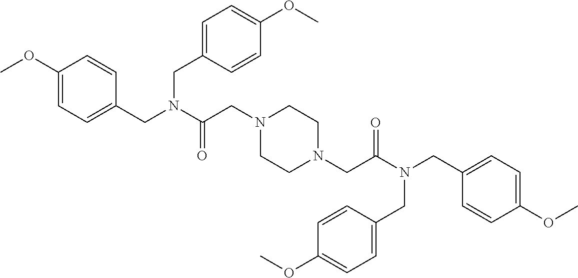 | Log P: 4.6, CLogP: 5.048 | Group I | Not Assayed |
| 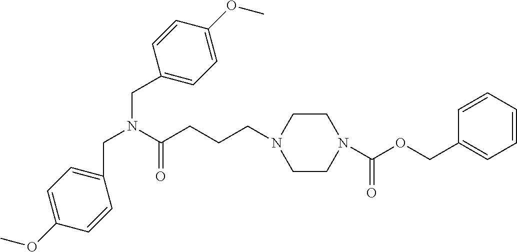 [SMILES: COc1ccc(CN(Cc2ccc(OC)cc2)C(=O)CCCN2CCN(C(=O)OCc3ccccc3)CC2)cc1] | Log P: 4.46, CLogP: 6.1365 | Group I | 3 fold increased response |
| 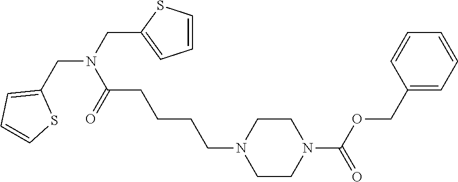 [SMILES: O=C(CCCCN1CCN(C(=O)OCc2ccccc2)CC1)N(Cc1cccs1)Cc1cccs1] | Log P: 5.09, CLogP: 5.356 | Group I | 0.9 fold response |
| 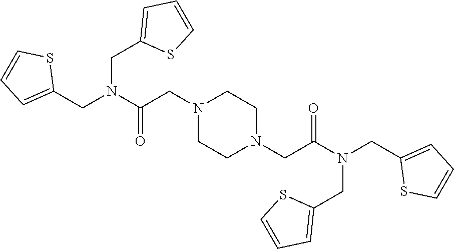 [SMILES: O=C(CN1CCN(CC(=O)N(Cc2cccs2)Cc2cccs2)CC1)N(Cc1cccs1)Cc1cccs1] | Log P: 5.03, CLogP: 3.956 | Group II | 0.4 fold response |
| 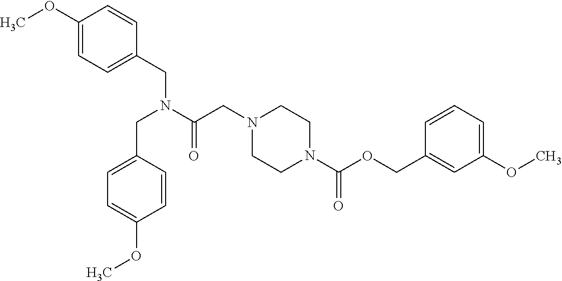 | Log P: 3.76, CLogP: 5.8628 | Group II | 2.2 fold increased response |
| 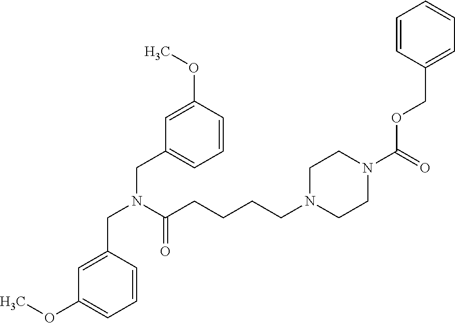 [SMILES: COc1cccc(CN(Cc2cccc(OC)c2)C(=O)CCCCN2CCN(C(=O)OCc3ccccc3)CC2)c1] | Log P: 4.88, CLogP: 5.902 | Group II | 3.9 fold increased response |
| 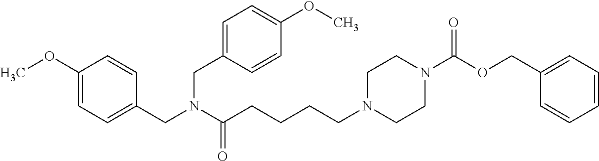 [SMILES: COc1ccc(CN(Cc2ccc(OC)cc2)C(=O)CCCCN2CCN(C(=O)OCc3ccccc3)CC2)cc1] | Log P: 4.88, CLogP: 5.902 | Group II | 4.3 fold increased response |
| 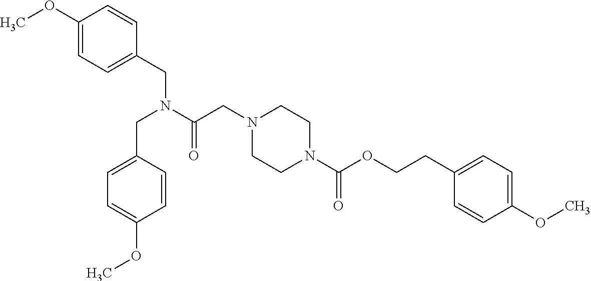 | Log P: 4.04, CLogP: 5.7322 | Group II | 4.6 fold increased response |
| 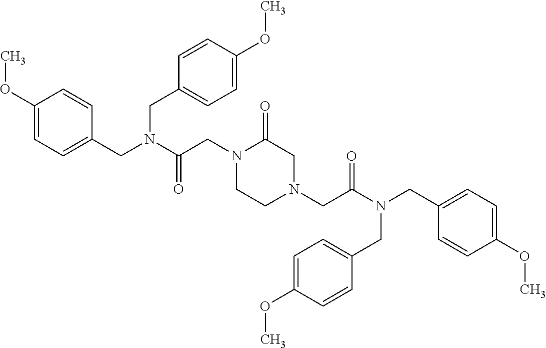 [SMILES: COc1ccc(CN(Cc2ccc(OC)cc2)C(=O)CN2CCN(CC(=O)N(Cc3ccc(OC)cc3)Cc3ccc(OC)cc3)C(=O)C2)cc1] | Log P: 3.69, CLogP: 6.4647 | Group I | 5.9 fold increased response |
| 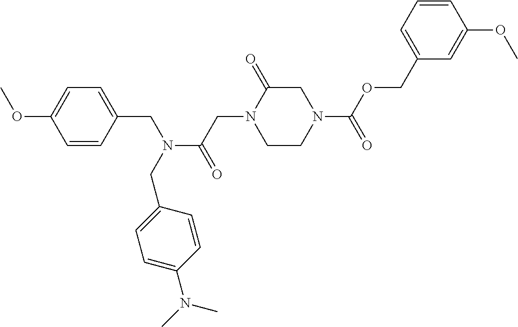 | Log P: 3.26, CLogP: 5.9722 | Group II | 4.2 fold increased response |
| 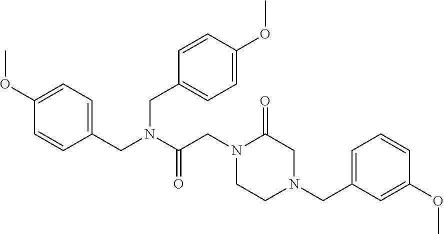 [SMILES: COc1ccc(CN(Cc2ccc(OC)cc2)C(=O)CN2CCN(Cc3cccc(OC)c3)CC2=O)cc1] | Log P: 2.99, CLogP: 5.2598 | Group IV | 1.1 fold increased response |
| 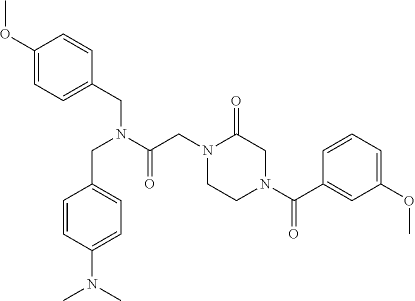 [SMILES: COc1ccc(CN(Cc2ccc(N(C)C)cc2)C(=O)CN2CCN(C(=O)c3cccc(OC)c3)CC2=O)cc1] | Log P: 2.84, CLogP: 4.8325 | Group IV | 1.2 fold increased response |
| 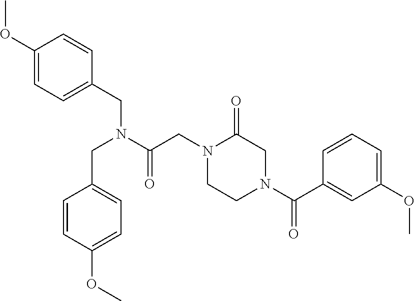 [SMILES: COc1ccc(CN(Cc2ccc(OC)cc2)C(=O)CN2CCN(C(=O)c3cccc(OC)c3)CC2=O)cc1] | Log P: 2.43, CLogP: 4.5865 | Group IV | 0.6 fold response |
| 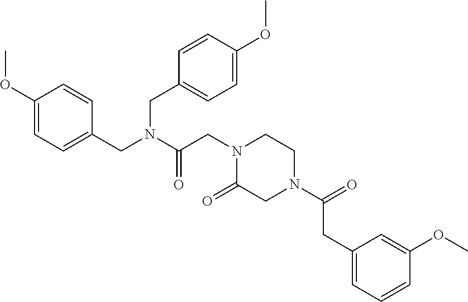 [SMILES: COc1ccc(CN(Cc2ccc(OC)cc2)C(=O)CN2CCN(C(=O)Cc3cccc(OC)c3)CC2=O)cc1] | Log P: 2.37, CLogP: 4.7722 | Group III | 0.5 fold response |
| 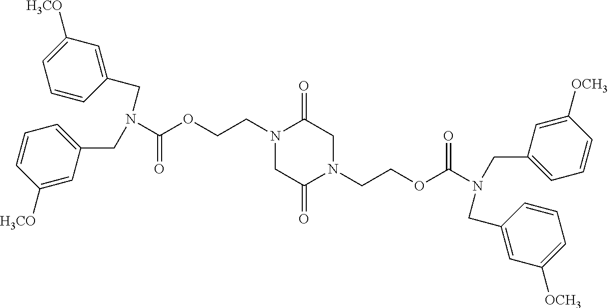 [SMILES: COc1cccc(CN(Cc2cccc(OC)c2)C(=O)OCCN2CC(=O)N(CCOC(=O)N(Cc3cccc(OC)c3)Cc3cccc(OC)c3)CC2=O)c1] | Log P: 6.25** | Group I | 3.4 fold increased response |
| 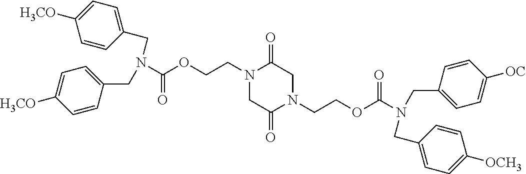 | Log P: 6.25** | Group I | 7.8 fold increased response |
| 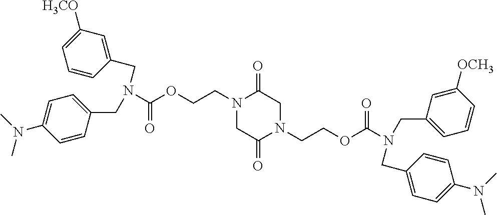 | Log P: 6.64** | Group I | 7.8 fold increased response |
| 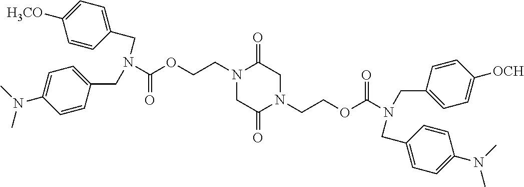 | Log P: 6.64** | Group I | 9.9 fold increased response |
| 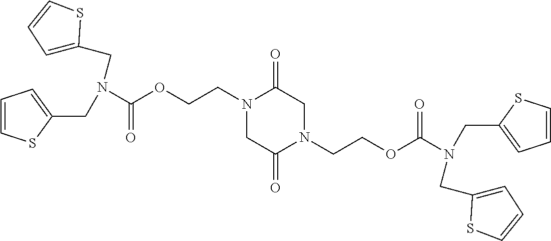 [SMILES: O=C1CN(CCOC(=O)N(Cc2cccs2)Cc2cccs2)C(=O)CN1CCOC(=O)N(Cc1cccs1)Cc1cccs1] | Log P: 4.74, CLogP: 6.6385 | Group II | 2 fold increased response |
| 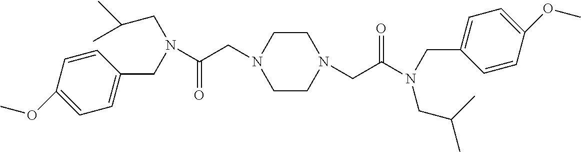 | Log P: 3.83, CLogP: 4.852 | Group IV | 0.9 fold response |
| 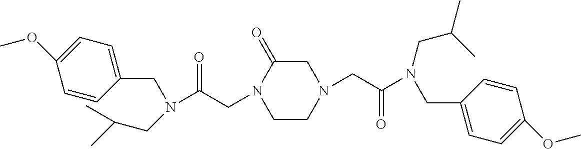 | Log P: 2.92, CLogP: 6.2687 | Group III | 0.19 fold response |
| 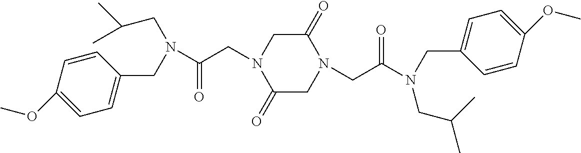 | Log P: 2.01, CLogP: 5.6976 | Group III | 0.15 fold response |
| 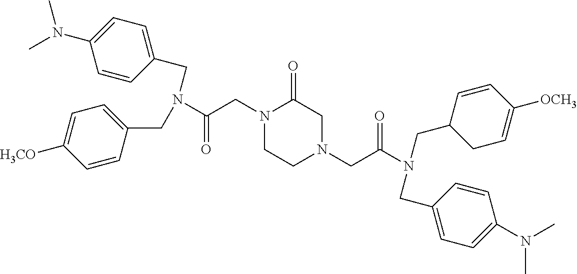 | Log P: 7.42** | Group III | 5.2 fold increased response |
| 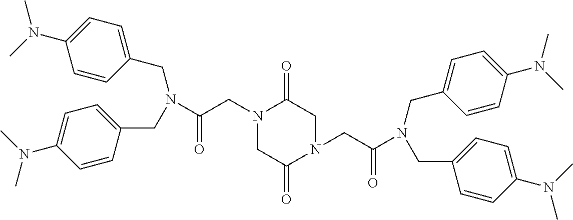 | Log P: 7.02** | Group I | Not Assayed |

All Log P values were obtained using Chemdraw Ultra® version 12.0 unless the structure exceeded the capability of the program to calculate a log P. The ** Indicates that Log P values were calculated using ACD/Chemsketch® Release 12.00 Version 12.01 in the alternative when otherwise not calculable using Chemdraw Ultra.

The forgoing exemplified structures (chart) generally had Log P values less than about 6 with many being in the 2-5 range with the expectation of improved water solubility profile when compared to the log P of the THI 0019 compound or THI0349. In an embodiment, the log P value ranges from about 2 to about 5. It is also noted that in the few embodiments where the log P values exceed 7, most of those compounds bore ionizable groups (that are not reflected in the log P calculation). Generally, greater potency was observed over that of THI0019 while increasing the expectation of improved water solubility.

While many embodiments of the disclosed agonist compounds are synthetic compounds of Formula I, in some embodiments an agonist compound is formed by in vivo conversion of a precursor compound to a disclosed compound. For example, a disclosed compound may exist as a stereoisomer wherein asymmetric or chiral centers are present. These stereoisomers are “R” or “S” depending on the configuration of substituents around the chiral carbon atom. The present invention contemplates various stereoisomers and mixtures thereof. Stereoisomers include enantiomers and diastereomers, and mixtures of enantiomers or diastereomers. Individual stereoisomers of some agonist compounds may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Various embodiments of the disclosed agonist compounds may exist in unsolvated or solvated forms, including hydrated forms, such as hemi-hydrates. In general, the solvated forms, with pharmaceutically acceptable solvents such as water and ethanol among others are equivalent to the unsolvated forms for the purposes of this disclosure. Pharmaceutical compositions containing the disclosed agonist compounds are described below.

Without being limited hereto, other embodiments of the disclosed agonist may also use related substitutes for the central ring such as:

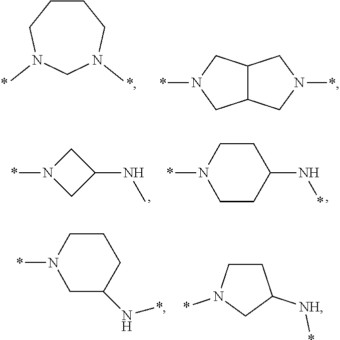

wherein the * indicates the attachment point.

### Pharmaceutical Compositions

The compounds described herein may be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66:1 et seq. The salts may be prepared in situ during the final isolation and purification of the compounds or separately by reacting a free base function with a suitable inorganic or organic acid. Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

In some embodiments, basic addition salts are prepared in situ during the final isolation and purification of a disclosed compound by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

Dosage forms for topical administration of a disclosed agonist compound include powders, sprays, ointments and inhalants. The active compound is mixed under sterile conditions with a pharmaceutically acceptable carrier and any needed preservatives, buffers or propellants which can be required. Ophthalmic formulations, eye ointments, powders and solutions are also contemplated in some embodiments.

Actual dosage levels of active ingredients in the pharmaceutical compositions may be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

In certain embodiments, the integrin agonists of Formula I and other components, when present, may be administered separately, collectively or concurrently, via any parenteral or non-parenteral administration procedure, wherein the other components include, without limitation, therapeutic antibodies, check point inhibitors, treated and/or untreated effector cells, antigens, adjuvants, excipients, stem cells, progenitor cells, other integrin expressing cells, or any combination thereof.

In other embodiments, the integrin agonists of Formula I and the other components are non-parenterally administered.

In other embodiments, the integrin agonists of Formula I and the other components are parenterally administered.

In other embodiments, the integrin agonists of Formula I non-parenterally administered before, during, and/or after administration of other components, wherein the administration of the other components may be via any acceptable administration procedure such as, without limitation, systemic administration, oral administration, IV administration, arterial administration, direct into tissue administration, any other administration procedure or any combination thereof.

When used for various therapeutic treatments, a therapeutically effective amount of one or more of the disclosed compounds may be employed in a pure form or, where such forms exist, in pharmaceutically acceptable salt, ester or pro-drug form. In some embodiments, the compound is administered as a pharmaceutical composition containing the compound of interest in combination with one or more pharmaceutically acceptable excipients. The phrase “therapeutically effective amount” of a disclosed agonist compound means a sufficient amount of the compound to treat disorders, at a reasonable benefit/risk ratio applicable to any medical treatment. It will be understood, however, that the total daily usage of the disclosed compounds and compositions will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the disclosed compounds administered to a human or lower animal may range from about 0.0001 to about 1000 mg/kg/day. For purposes of oral administration, in some embodiments, doses are in the range of from about 0.001 to about 5 mg/kg/day. If desired, the effective daily dose can be divided into multiple doses for purposes of administration; consequently, single dose compositions may contain such amounts or submultiples thereof to make up the daily dose.

In other embodiments, a dose of the integrin agonists of Formula (I) are adapted to produce an effective concentration measure in molarity units of the integrin agonists in a patient's blood or at a site of action of a patient such as a tumor stroma, a bone marrow stroma, or a site of any other treatable disease or malady. The effective concentration is generally between about 1 fM and about 300 μM, between about 1 nM and about 300 μM, between about 10 nM and about 300 μM, or between about 25 nM and 300 μM.

In some instances, a pharmaceutical composition comprises one or more of the disclosed compounds formulated together with one or more non-toxic pharmaceutically acceptable carriers. The pharmaceutical compositions may be specially formulated for oral administration in solid or liquid form, for parenteral injection or for rectal administration.

The disclosed pharmaceutical compositions may be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion. In some implementations, a pharmaceutical composition comprises a disclosed compound and a physiologically tolerable or acceptable diluent, carrier, adjuvant or vehicle, which are collectively referred to herein as diluents, for parenteral injection, for intranasal delivery, for oral administration in solid or liquid form, for rectal or topical administration, or the like.

In some instances, a composition is delivered through a catheter for local delivery at a target site, via an intracoronary stent (a tubular device composed of a fine wire mesh), or via a biodegradable polymer. In some embodiments, an agonist compound is complexed to a ligand such as an antibody, for targeted delivery.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. Proper fluidity can be maintained, for example, by the use of coating materials such as lecithin, by the maintenance of the required particle size in the case of dispersions and by the use of surfactants.

In some embodiments, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline foam. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients. Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents. Compositions for rectal or vaginal administration are preferably suppositories which can be prepared by mixing one or more of the disclosed compounds with suitable non-irritating excipients or carriers such as cocoa butter, polyethylene glycol or a suppository wax which are solid at room temperature but liquid at body temperature and therefore melt in the rectum or vaginal cavity and release the active compound.

For some applications, a disclosed compound is administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. In some embodiments, a composition in liposome form contains, in addition to a disclosed agonist compound, stabilizers, preservatives, excipients and the like. The preferred lipids are natural and synthetic phospholipids and phosphatidyl cholines (lecithins) used separately or together. Methods of forming liposomes are known in the art. See, for example, Prescott, Ed., Methods in cell Biology, Volume XIV, Academic Press, New York, N. Y. (1976), p. 33 et seq.

The term “pharmaceutically acceptable pro-drugs” as used herein represents those pro-drugs of the disclosed compounds which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the disclosed compounds. Pro-drugs according to certain embodiments may be rapidly transformed in vivo to the parent compound of the above formula, for example, by hydrolysis in blood.

While the preferred embodiments have been shown and described, modifications thereof can be made by one skilled in the art without departing from the spirit and teachings of the disclosed subject matter. The embodiments described herein are exemplary and are non-limiting. Many variations and modifications of the subject matter disclosed herein are possible and are within the scope of this disclosure. Accordingly, the scope of protection is not limited by the description set out above, but is only limited by the claims which follow, that scope including all equivalents of the subject matter of the claims. All patents, patent applications and publications cited herein are hereby incorporated herein by reference to the extent that they provide materials, methods and explanatory details supplementary to those set forth herein.

1. A compound of formula I, 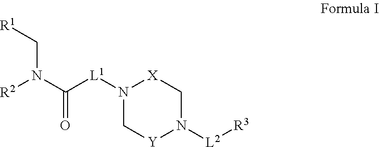
wherein R¹ is an aryl ring;
R² comprises an aryl group, an aralkyl group, or a lower alkyl group;
L¹ is a linker selected from a group consisting essentially of —(CH₂)ₙ—, —O(CH₂)ₙ—, and —(CH₂)ₙO(CH₂)ₚ—;
L² is a linker selected from a group consisting essentially of —CO—, —CO(CH₂)ₘ, —COO(CH₂)ₘ—, —(CH₂)ₘ—, —(CH₂)ₘO—, and —(CH₂)ₘO(CH₂)_{q}—;
R³ is selected from a group consisting essentially of aryl, heterocyclyl, CONR⁴R⁵, and —COR⁶;
X and Y are independently selected from —CH₂— and —C(O)—;
n is an integer of from 1 to 4;
m, p, and q, each of which when present, are independently an integer of from 1 to 2;
R⁴ and R⁵, when present, are independently selected from a group consisting essentially of hydrogen, a lower alkyl group and an aralkyl group;
R⁶, when present, is a heterocyclic ring;
When present, each R¹ and R² may be unsubstituted or substituted with a substituent selected from the group consisting essentially of a lower alkyl group, an alkoxy group, a hydroxyalkyl group, —OH, an alkoxyalkyl group, a (C₁-C₃ alkyl)₂amino group, an alkoxyalkoxy group, a cycloalkyl, cycloalkylalkyl group, an aryl group, a heterocyclyl group, an alkylaryl group, an aralkyl group, an alkylheterocyclyl group, and a heterocyclylalkyl group;
R³, R⁴, R⁵ and R⁶ may be unsubstituted or substituted with a substituent selected from the group consisting essentially of hydroxy, alkoxy, dialkylamino, halogen, a lower alkyl group, a hydroxyalkyl group, an aliphatic acyl group, —CF₃, oxo, —CN, an alkoxyalkyl group, a (C₁-C₃ alkyl)₂amino group, an alkoxyalkoxy group, a cycloalkyl group, a cycloalkylalkyl group, an aryl group, a heterocyclyl group, an alkylaryl group, an aralkyl group, an alkylheterocyclyl group, a heterocyclylalkyl group, and an aryloxyalkyl group;
and pharmaceutically acceptable salts thereof.

2. The compound of claim 1, wherein R¹ is selected from the group consisting essentially of substituted phenyl, and substituted or unsubstituted heteroaromatics selected from the group consisting essentially of: thienyl, oxazolyl, isoxazolyl, pyrrolyl and pyridyl.

3. The compound of claim 1, wherein R³ is selected from the group consisting essentially of: 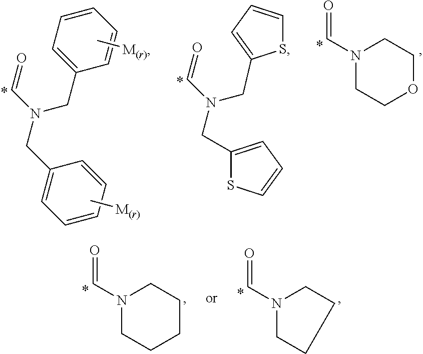
the asterisk * represents the attachment to L²;
wherein each M, when present, is selected from the group consisting essentially of hydroxy, alkoxy, dialkylamino, halogen, and alkyl; and
each r, when resent, is an integer from 1-2.

4. The compound of claim 1, selected from the group consisting essentially of: 2,2′-((piperazine-1,4-diylbis(ethane-2,1-diyl))bis(oxy))bis(N,N-bis(thiophen-2-ylmethyl)acetamide), piperazine-1,4-diylbis(ethane-2,1-diyl) bis(bis(3-methoxybenzyl)carbamate); benzyl 4-(2-((4-(dimethylamino)benzyl)(4-methoxybenzyl)amino)-2-oxoethyl)piperazine-1-carboxylate; 2,2′-(2,5-dioxopiperazine-1,4-diyl)bis(N,N-bis(thiophen-2-ylmethyl)acetamide); benzyl 4-(2-((4-hydroxybenzyl)(4-methoxybenzyl)amino)-2-oxoethyl)piperazine-1-carboxylate; benzyl 4-(2-((4-(dimethylamino)benzyl)(4-hydroxybenzyl)amino)-2-oxoethyl)piperazine-1-carboxylate dihydrochloride; benzyl 4-(2-(bis(4-hydroxybenzyl)amino)-2-oxoethyl)piperazine-1-carboxylate; benzyl 4-(2-(bis(4-methoxybenzyl)amino)-2-oxoethyl)piperazine-1-carboxylate; benzyl 4-(2-(bis(3-methoxybenzyl)amino)-2-oxoethyl)piperazine-1-carboxylate; piperazine-1,4-diylbis(ethane-2,1-diyl)bis(4-(dimethylamino)benzyl (4-methoxybenzyl)carbamate); 2,2′-(2,5-dioxopiperazine-1,4-diyl)bis(N-(3-(dimethylamino)benzyl)-N-(4-methoxybenzyl)acetamide); 2,2′-(2,5-dioxopiperazine-1,4-diyl)bis(N,N-bis(3-methoxybenzyl)acetamide); 2,2′-(piperazine-1,4-diyl)bis(N,N-bis(4-methoxybenzyl)acetamide); benzyl 4-(4-(bis(4-methoxybenzyl)amino)-4-oxobutyl)piperazine-1-carboxylate; benzyl 4-(5-(bis(thiophen-2-ylmethyl)amino)-5-oxopentyl)piperazine-1-carboxylate; 2,2′-(piperazine-1,4-diyl)bis(N,N-bis(thiophen-2-ylmethyl)acetamide); 3-methoxybenzyl 4-(2-(bis(4-methoxybenzyl)amino)-2-oxoethyl)piperazine-1-carboxylate; benzyl 4-(5-(bis(3-methoxybenzyl)amino)-5-oxopentyl)piperazine-1-carboxylate; benzyl 4-(5-(bis(4-methoxybenzyl)amino)-5-oxopentyl)piperazine-1-carboxylate; 4-methoxyphenethyl 4-(2-(bis(4-methoxybenzyl)amino)-2-oxoethyl)piperazine-1-carboxylate; 2,2′-(2-oxopiperazine-1,4-diyl)bis(N,N-bis(4-methoxybenzyl)acetamide); 3-methoxybenzyl 4-(2-((4-(dimethylamino)benzyl)(4-methoxybenzyl)amino)-2-oxoethyl)-3-oxopiperazine-1-carboxylate; N,N-bis(4-methoxybenzyl)-2-(4-(3-methoxybenzyl)-2-oxopiperazin-1-yl)acetamide; N-(4-(dimethylamino)benzyl)-2-(4-(3-methoxybenzoyl)-2-oxopiperazin-1-yl)-N-(4-methoxybenzyl)acetamide; 2-(4-(3-methoxybenzoyl)-2-oxopiperazin-1-yl)-N,N-bis(4-methoxybenzyl)acetamide; N,N-bis(4-methoxybenzyl)-2-(4-(2-(3-methoxyphenyl)acetyl)-2-oxopiperazin-1-yl)acetamide; (2,5-dioxopiperazine-1,4-diyl)bis(ethane-2,1-diyl) bis(bis(3-methoxybenzyl)carbamate); (2,5-dioxopiperazine-1,4-diyl)bis(ethane-2,1-diyl) bis(bis(4-methoxybenzyl)carbamate); (2,5-dioxopiperazine-1,4-diyl)bis(ethane-2,1-diyl) bis(4-(dimethylamino)benzyl (3-methoxybenzyl)carbamate); (2,5-dioxopiperazine-1,4-diyl)bis(ethane-2,1-diyl)bis(4-(dimethylamino)benzyl (4-methoxybenzyl)carbamate); (2,5-dioxopiperazine-1,4-diyl)bis(ethane-2,1-diyl) bis(bis(thiophen-2-ylmethyl)carbamate); 2,2′-(piperazine-1,4-diyl)bis(N-isobutyl-N-(4-methoxybenzyl)acetamide); 2,2′-(2-oxopiperazine-1,4-diyl)bis(N-isobutyl-N-(4-methoxybenzyl)acetamide); 2,2′-(2,5-dioxopiperazine-1,4-diyl)bis(N-isobutyl-N-(4-methoxybenzyl)acetamide); 2,2′-(2-oxopiperazine-1,4-diyl)bis(N-(4-(dimethylamino)benzyl)-N-(4-methoxybenzyl)acetamide); 2,2′-(2,5-dioxopiperazine-1,4-diyl)bis(N,N-bis(4-(dimethylamino)benzyl)acetamide), solvates thereof, precursors thereof and hydrates thereof.

5. The compound of claim 1, wherein the log P is less than about 6.

6. The compound of claim 1 characterized by Formula I, 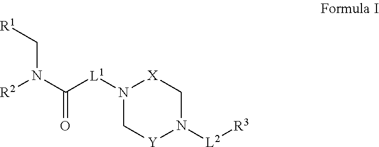
wherein R¹ is an aryl ring;
R² comprises an aryl group, an aralkyl group, or a lower alkyl group;
L¹ is a linker selected from a group consisting essentially of —(CH₂)ₙ—, —O(CH₂)ₙ—, and —(CH₂)ₙO(CH₂)ₚ—;
L² is a linker selected from a group consisting essentially of —CO—, —CO(CH₂)ₘ, —COO(CH₂)ₘ—, —(CH₂)ₘ—, —(CH₂)ₘO—, and —(CH₂)ₘO(CH₂)_{q}—;
R³ is selected from a group consisting essentially of aryl, heterocyclyl, CONR⁴R⁵, and —COR⁶;
X and Y are independently selected from —CH₂— and —C(O)—;
n is an integer of from 1 to 4;
m, p, and q, each of which when present, are independently an integer of from 1 to 2
R⁴ and R⁵, when present, are independently selected from a group consisting essentially of hydrogen, a lower alkyl group and an aralkyl group;
R⁶, when present, is a heterocyclic ring;
when present, each R¹ and R² may be unsubstituted or substituted with a substituent selected from the group consisting essentially of a lower alkyl group, an alkoxy group, a hydroxyalkyl group, —OH, an alkoxyalkyl group, a (C₁-C₃ alkyl)₂amino group, an alkoxyalkoxy group, a cycloalkyl, cycloalkylalkyl group, an aryl group, a heterocyclyl group, an alkylaryl group, an aralkyl group, an alkylheterocyclyl group, and a heterocyclylalkyl group;
R³, R⁴, R⁵ and R⁶ may be unsubstituted or substituted with a substituent selected from the group consisting essentially of hydroxy, alkoxy, dialkylamino, halogen, a lower alkyl group, a hydroxyalkyl group, an aliphatic acyl group, —CF₃, oxo, —CN, an alkoxyalkyl group, a (C₁-C₃ alkyl)₂amino group, an alkoxyalkoxy group, a cycloalkyl group, a cycloalkylalkyl group, an aryl group, a heterocyclyl group, an alkylaryl group, an aralkyl group, an alkylheterocyclyl group, a heterocyclylalkyl group, and an aryloxyalkyl group;
and a pharmaceutically acceptable carrier.

7. A medicament for use in the treatment of any condition susceptible of being improved or prevented by the selective occupation of an integrin receptor, comprising the compound of Formula I, 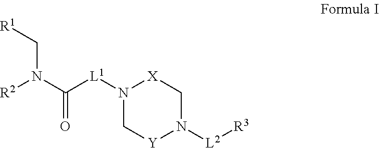
wherein R¹ is an aryl ring;
R² comprises an aryl group, an aralkyl group, or a lower alkyl group;
L¹ is a linker selected from a group consisting essentially of —(CH₂)ₙ—, —O(CH₂)ₙ—, and —(CH₂)ₙO(CH₂)ₚ—;
L² is a linker selected from a group consisting essentially of —CO—, —CO(CH₂)ₘ, —COO(CH₂)ₘ—, —(CH₂)ₘ—, —(CH₂)ₘO—, and —(CH₂)ₘO(CH₂)_{q}—;
R³ is selected from a group consisting essentially of aryl, heterocyclyl, CONR⁴R⁵, and —COR⁶;
X and Y are independently selected from —CH₂— and —C(O)—;
n is an integer of from 1 to 4;
m, p, and q, each of which when present, are independently an integer of from 1 to 2;
R⁴ and R⁵, when present, are independently selected from a group consisting essentially of hydrogen, a lower alkyl group and an aralkyl group;
R⁶, when present, is a heterocyclic ring;
when present, each R¹ and R² may be unsubstituted or substituted with a substituent selected from the group consisting essentially of a lower alkyl group, an alkoxy group, a hydroxyalkyl group, —OH, an alkoxyalkyl group, a (C₁-C₃ alkyl)₂amino group, an alkoxyalkoxy group, a cycloalkyl, cycloalkylalkyl group, an aryl group, a heterocyclyl group, an alkylaryl group, an aralkyl group, an alkylheterocyclyl group, and a heterocyclylalkyl group;
R³, R⁴, R⁵ and R⁶ may be unsubstituted or substituted with a substituent selected from the group consisting essentially of hydroxy, alkoxy, dialkylamino, halogen, a lower alkyl group, a hydroxyalkyl group, an aliphatic acyl group, —CF₃, oxo, —CN, an alkoxyalkyl group, a (C₁-C₃ alkyl)₂amino group, an alkoxyalkoxy group, a cycloalkyl group, a cycloalkylalkyl group, an aryl group, a heterocyclyl group, an alkylaryl group, an aralkyl group, an alkylheterocyclyl group, a heterocyclylalkyl group, and an aryloxyalkyl group;
and pharmaceutically acceptable salts thereof.

8. The medicament of claim 7, wherein the integrin is selected from the group consisting essentially of α4β1, α5β1, α4β7, and αLβ2.

9. The medicament of claim 7, further comprising a pharmaceutically acceptable excipient, a pharmaceutically acceptable carrier or both.

10. The medicament of claim 7, comprising a liposome comprising a compound selected from the group consisting essentially of: 2,2′-((piperazine-1,4-diylbis(ethane-2,1-diyl))bis(oxy))bis(N,N-bis(thiophen-2-ylmethyl)acetamide), piperazine-1,4-diylbis(ethane-2,1-diyl) bis(bis(3-methoxybenzyl)carbamate); benzyl 4-(2-((4-(dimethylamino)benzyl)(4-methoxybenzyl)amino)-2-oxoethyl)piperazine-1-carboxylate; 2,2′-(2,5-dioxopiperazine-1,4-diyl)bis(N,N-bis(thiophen-2-ylmethyl)acetamide); benzyl 4-(2-((4-hydroxybenzyl)(4-methoxybenzyl)amino)-2-oxoethyl)piperazine-1-carboxylate; benzyl 4-(2-((4-(dimethylamino)benzyl)(4-hydroxybenzyl)amino)-2-oxoethyl)piperazine-1-carboxylate dihydrochloride; benzyl 4-(2-(bis(4-hydroxybenzyl)amino)-2-oxoethyl)piperazine-1-carboxylate; benzyl 4-(2-(bis(4-methoxybenzyl)amino)-2-oxoethyl)piperazine-1-carboxylate; benzyl 4-(2-(bis(3-methoxybenzyl)amino)-2-oxoethyl)piperazine-1-carboxylate; piperazine-1,4-diylbis(ethane-2,1-diyl) bis(4-(dimethylamino)benzyl (4-methoxybenzyl)carbamate); 2,2′-(2,5-dioxopiperazine-1,4-diyl)bis(N-(3-(dimethylamino)benzyl)-N-(4-methoxybenzyl)acetamide); 2,2′-(2,5-dioxopiperazine-1,4-diyl)bis(N,N-bis(3-methoxybenzyl)acetamide); 2,2′-(piperazine-1,4-diyl)bis(N,N-bis(4-methoxybenzyl)acetamide); benzyl 4-(4-(bis(4-methoxybenzyl)amino)-4-oxobutyl)piperazine-1-carboxylate; benzyl 4-(5-(bis(thiophen-2-ylmethyl)amino)-5-oxopentyl)piperazine-1-carboxylate; 2,2′-(piperazine-1,4-diyl)bis(N,N-bis(thiophen-2-ylmethyl)acetamide); 3-methoxybenzyl 4-(2-(bis(4-methoxybenzyl)amino)-2-oxoethyl)piperazine-1-carboxylate; benzyl 4-(5-(bis(3-methoxybenzyl)amino)-5-oxopentyl)piperazine-1-carboxylate; benzyl 4-(5-(bis(4-methoxybenzyl)amino)-5-oxopentyl)piperazine-1-carboxylate; 4-methoxyphenethyl 4-(2-(bis(4-methoxybenzyl)amino)-2-oxoethyl)piperazine-1-carboxylate; 2,2′-(2-oxopiperazine-1,4-diyl)bis(N,N-bis(4-methoxybenzyl)acetamide); 3-methoxybenzyl 4-(2-((4-(dimethylamino)benzyl)(4-methoxybenzyl)amino)-2-oxoethyl)-3-oxopiperazine-1-carboxylate; N,N-bis(4-methoxybenzyl)-2-(4-(3-methoxybenzyl)-2-oxopiperazin-1-yl)acetamide; N-(4-(dimethylamino)benzyl)-2-(4-(3-methoxybenzoyl)-2-oxopiperazin-1-yl)-N-(4-methoxybenzyl)acetamide; 2-(4-(3-methoxybenzoyl)-2-oxopiperazin-1-yl)-N,N-bis(4-methoxybenzyl)acetamide; N,N-bis(4-methoxybenzyl)-2-(4-(2-(3-methoxyphenyl)acetyl)-2-oxopiperazin-1-yl)acetamide; (2,5-dioxopiperazine-1,4-diyl)bis(ethane-2,1-diyl) bis(bis(3-methoxybenzyl)carbamate); (2,5-dioxopiperazine-1,4-diyl)bis(ethane-2,1-diyl) bis(bis(4-methoxybenzyl)carbamate); (2,5-dioxopiperazine-1,4-diyl)bis(ethane-2,1-diyl) bis(4-(dimethylamino)benzyl (3-methoxybenzyl)carbamate); (2,5-dioxopiperazine-1,4-diyl)bis(ethane-2,1-diyl) bis(4-(dimethylamino)benzyl (4-methoxybenzyl)carbamate); (2,5-dioxopiperazine-1,4-diyl)bis(ethane-2,1-diyl) bis(bis(thiophen-2-ylmethyl)carbamate); 2,2′-(piperazine-1,4-diyl)bis(N-isobutyl-N-(4-methoxybenzyl)acetamide); 2,2′-(2-oxopiperazine-1,4-diyl)bis(N-isobutyl-N-(4-methoxybenzyl)acetamide); 2,2′-(2,5-dioxopiperazine-1,4-diyl)bis(N-isobutyl-N-(4-methoxybenzyl)acetamide); 2,2′-(2-oxopiperazine-1,4-diyl)bis(N-(4-(dimethylamino)benzyl)-N-(4-methoxybenzyl)acetamide); 2,2′-(2,5-dioxopiperazine-1,4-diyl)bis(N,N-bis(4-(dimethylamino)benzyl)acetamide), solvates thereof, precursors thereof and hydrates thereof.

11. The medicament of claim 7 which is an ophthalmic formulation comprising a lymphocyte function-associated antigen-1 (LFA-1) agonist comprising the compound of Formula I, 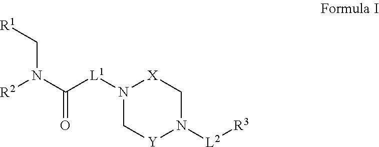
wherein R¹ is an aryl ring;
R² comprises an aryl group, an aralkyl group, or a lower alkyl group;
L¹ is a linker selected from a group consisting essentially of —(CH₂)ₙ—, —O(CH₂)ₙ—, and —(CH₂)ₙO(CH₂)ₚ—;
L² is a linker selected from a group consisting essentially of —CO—, —CO(CH₂)ₘ, —COO(CH₂)ₘ—, —(CH₂)ₘ—, —(CH₂)ₘO—, and —(CH₂)ₘO(CH₂)_{q}—;
R³ is selected from a group consisting essentially of aryl, heterocyclyl, CONR⁴R⁵, and —COR⁶;
X and Y are independently selected from —CH₂— and —C(O)—;
n is an integer of from 1 to 4;
m, p, and q, each of which when present, are independently an integer of from 1 to 2;
R⁴ and R⁵, when present, are independently selected from a group consisting essentially of hydrogen, a lower alkyl group and an aralkyl group;
R⁶, when present, is a heterocyclic ring;
when present, each R¹ and R² may be unsubstituted or substituted with a substituent selected from the group consisting essentially of a lower alkyl group, an alkoxy group, a hydroxyalkyl group, —OH, an alkoxyalkyl group, a (C₁-C₃ alkyl)₂amino group, an alkoxyalkoxy group, a cycloalkyl, cycloalkylalkyl group, an aryl group, a heterocyclyl group, an alkylaryl group, an aralkyl group, an alkylheterocyclyl group, and a heterocyclylalkyl group;
R³, R⁴, R⁵ and R⁶ may be unsubstituted or substituted with a substituent selected from the group consisting essentially of hydroxy, alkoxy, dialkylamino, halogen, a lower alkyl group, a hydroxyalkyl group, an aliphatic acyl group, —CF₃, oxo, —CN, an alkoxyalkyl group, a (C₁-C₃ alkyl)₂amino group, an alkoxyalkoxy group, a cycloalkyl group, a cycloalkylalkyl group, an aryl group, a heterocyclyl group, an alkylaryl group, an aralkyl group, an alkylheterocyclyl group, a heterocyclylalkyl group, and an aryloxyalkyl group;
and pharmaceutically acceptable salts thereof.

12. The medicament of claim 11, wherein R³ is selected from the group consisting essentially of: 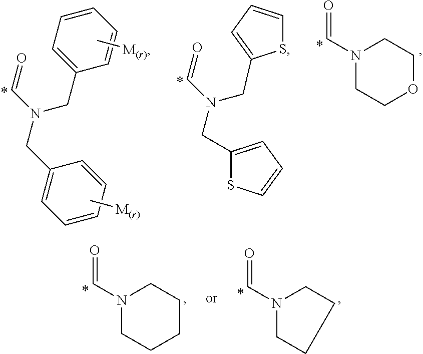
the asterisk * represents the attachment to L²;
wherein each M, when present, is selected from the groups consisting essentially of hydroxy, alkoxy, dialkylamino, halogen, and alkyl; and
each r, when present, is an integer from 1-2.

13. A complex formed between (i) an integrin expressing cell and an integrin agonist; and/or (ii) an integrin binding protein wherein the integrin agonist has general Formula I 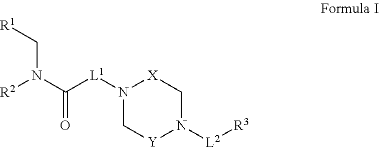
wherein R¹ is an aryl ring;
R² comprises an aryl group, an aralkyl group, or a lower alkyl group;
L¹ is a linker selected from a group consisting essentially of —(CH₂)ₙ—, —O(CH₂)ₙ—, and —(CH₂)ₙO(CH₂)ₚ—;
L² is a linker selected from a group consisting essentially of —CO—, —CO(CH₂)ₘ, —COO(CH₂)ₘ—, —(CH₂)ₘ—, —(CH₂)ₘO—, and —(CH₂)ₘO(CH₂)_{q}—;
R³ is selected from a group consisting essentially of aryl, heterocyclyl, CONR⁴R⁵, and —COR⁶;
X and Y are independently selected from —CH₂— and —C(O)—;
n is an integer of from 1 to 4;
m, p, and q, each of which when present, are independently an integer of from 1 to 2
R⁴ and R⁵, when present, are independently selected from a group consisting essentially of hydrogen, a lower alkyl group and an aralkyl group;
R⁶, when present, is a heterocyclic ring;
when present, each R¹ and R² may be unsubstituted or substituted with a substituent selected from the group consisting essentially of a lower alkyl group, an alkoxy group, a hydroxyalkyl group, —OH, an alkoxyalkyl group, a (C₁-C₃ alkyl)₂amino group, an alkoxyalkoxy group, a cycloalkyl, cycloalkylalkyl group, an aryl group, a heterocyclyl group, an alkylaryl group, an aralkyl group, an alkylheterocyclyl group, and a heterocyclylalkyl group;
R³, R⁴, R⁵ and R⁶ may be unsubstituted or substituted with a substituent selected from the group consisting essentially of hydroxy, alkoxy, dialkylamino, halogen, a lower alkyl group, a hydroxyalkyl group, an aliphatic acyl group, —CF₃, oxo, —CN, an alkoxyalkyl group, a (C₁-C₃ alkyl)₂amino group, an alkoxyalkoxy group, a cycloalkyl group, a cycloalkylalkyl group, an aryl group, a heterocyclyl group, an alkylaryl group, an aralkyl group, an alkylheterocyclyl group, a heterocyclylalkyl group, and an aryloxyalkyl group;
and pharmaceutically acceptable salts thereof.

14. The complex of claim 13, wherein R³ is selected from the group consisting essentially of: 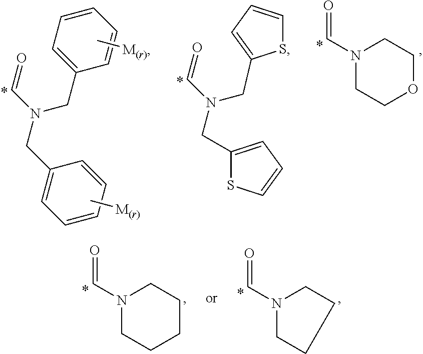
the asterisk * represents the attachment to L²;
wherein each M, when present, is selected from the groups consisting essentially of hydroxy, alkoxy, dialkylamino, halogen, and alkyl; and
each r, when present, is an integer from 1-2.

15. The complex of claim 13, wherein the integrin binding protein comprises vascular cell adhesion molecule-1 (VCAM 1), fibronectin, mucosal addressin cell adhesion molecule-1 (MAdCAM-1), intercellular adhesion molecule-1 (ICAM-1), intercellular adhesion molecule-2 (ICAM-2) or a combination thereof.

16. The complex of claim 13, wherein the integrin expressing cells comprise immune cells, embryonic stem cells, adult stem cells, progenitor cells, induced pluripotent stem cells, or a combination thereof.

17.-21. (canceled)
